(19) United States
(12) Patent Application Publication
(10) Pub. No.: US 2026/0124288 A1
(43) Pub. Date: May 7, 2026

(54) NOVEL VSV VIRUS FORMULATIONS

(71) Applicant: EMERGENT PRODUCT DEVELOPMENT GAITHERSBURG INC., Gaithersburg, MD (US)

(72) Inventors: Rockey BANDLISH, San Diego, CA (US); Ryan Paul DUFFIN, San Diego, CA (US); Amish PATEL, San Diego, CA (US); Christopher POON, Carlsbad, CA (US)

(21) Appl. No.: 19/117,838

(22) PCT Filed: Oct. 4, 2023
(86) PCT No.: PCT/US2023/075924

§ 371 Date: Apr. 2, 2025

Related U.S. Application Data
(60) Provisional application No. 63/413,062, filed on Oct. 4, 2022.

(51) Int. Cl. A61K 39/12; A61K 39/00; A61K 47/02; A61K 47/18; A61K 47/22; A61K 47/26; A61K 47/42; A61P 37/04

(52) U.S. Cl. CPC A61K 39/12; A61K 47/02; A61K 47/183; A61K 47/22; A61K 47/26; A61K 47/42; A61P 37/04; A61K 2039/5254; A61K 2039/5256; A61K 2039/54; A61K 2039/575

(57) ABSTRACT

Described herein are novel compositions comprising a virus or viral vector that may express viral glycoproteins, and methods of treatment using the same.

### TECHNICAL FIELD

Described herein are novel compositions comprising a Vesicular stomatitis virus (VSV) or VSV viral vectors that may express viral glycoproteins, and methods of treatment using the same.

### BACKGROUND OF THE INVENTION

Vesicular stomatitis virus (VSV), a member of the Rhabdoviridae family, has shown increasing promise as a cancer therapeutic (Cook et al. Blood Adv (2022) 6 (11): 3268-3279) and in live vaccine development as a viral vector. It is an enveloped, single-stranded RNA virus with a simple genome encoding a nucleocapsid, phosphoprotein, matrix protein, glycoprotein, and a large polymerase. Genetic manipulation of VSV makes it suitable for therapeutic and vaccine uses e.g., transgenes may be inserted in the VSV genome and the native glycoprotein may be replaced by the desired antigen. The VSV virus or VSV viral vector may by attenuated, e.g., limiting replication and decreasing its already low pathogenicity in humans. Expression of antigens on the surface of the viral particle allows stimulation of an immune response for long-term immunity. VSV vectors have the ability to produce significant amounts of viral particles, and a low seroprevalence in humans due to the natural host being livestock and insects.

Viral hemorrhagic fever is prevalent in Africa, and may be caused by several viruses. Lassa fever is a viral hemorrhagic disease that is prevalent in West Africa. Mainly found in Sierra Leone, Liberia, Guinea, and Nigeria, there is an increasing worry of spread to other countries. Lassa virus (LASV) belongs to the Arenaviridae family and is an enveloped, single-stranded, bipartite RNA virus that typically spreads to humans through exposure to its rodent host reservoir. Secondary transmission from person-to-person has also been identified in close contact dwellings or health care settings where infection control practices are limited.

Lassa virus (LASV) has an incubation period of 2 to 21 days with hospitalization rates of approximately 15% and a 1% fatality rate. Most infected are asymptomatic (˜80%) but those displaying symptoms may have fever, malaise, chest pain, sore throat, cough, difficulty breathing, and abdominal distress such as cramps, vomiting, or diarrhea. Those with severe manifestations may exhibit bleeding from mucosal surfaces, pleural and pericardial effusion, seizures, and unconsciousness. Individuals most susceptible to severe or fatal LASV are children and pregnant women. The greatest risk of getting the disease still results from rodent exposure such as households having a large numbers of rats or poor sanitary conditions often found in rural areas and migrant/refugee camps.

In addition to LASV, Marburg virus (MARV) and Ebola virus (EBOV) belong to the Filoviridae family and are single-stranded negative sense RNA viruses that cause severe hemorrhagic fever. Sudan virus (SUDV) is a species of EBOV. Transmission of these viruses is believed to occur via fruit bats, infected animals or humans. Symptoms of infection may include muscle aches, weakness, diarrhea, vomiting, and in some cases, severe bleeding and may result in a high mortality rate.

One remaining challenge related to VSV based vectors/vaccines, however, is stability. To this end, this disclosure addresses the need for a stable VSV based viral vaccine/vector formulation, e.g., a VSV based vaccine for viral hemorrhagic fever.

### INCORPORATION BY REFERENCE

All publications, patents, and patent applications mentioned in this specification are herein incorporated by reference with regard to their background teachings to the same extent as if each individual publication, patent, or patent application was specifically and individually indicated to be incorporated by reference.

### SUMMARY OF THE INVENTION

One embodiment described herein is a pharmaceutical composition comprising: (a) one or more sugars; (b) one or more amino acids; (c) one or more proteins; and (d) a virus or viral vector. In one aspect, the virus or viral vector is a Rhabdovirus vector. In one aspect, the virus or viral vector is a Vesicular Stomatitis Virus (VSV) vector. In another aspect, the virus or vector does not encode a homologous viral glycoprotein or fragment thereof. In one aspect, the vector encodes a heterologous viral transgene. In one aspect, the heterologous viral transgene is a Lassa virus transgene, a Ebola virus transgene, a Sudan virus transgene or a Marburg virus transgene. In other aspects, the heterologous viral transgene is located at position of 1 of the viral genome. In one aspect, a nucleocapsid N gene is located at position 4 of the viral genome.

In other aspects, the heterologous Lassa virus transgene encodes a Lassa virus glycoprotein or fragment thereof. In one aspect, the heterologous Lassa virus transgene encodes a Lassa virus immunogen. In one aspect, the heterologous viral transgene is a Marburg virus transgene. In one aspect, the heterologous Marburg virus transgene encodes a Marburg virus glycoprotein or fragment thereof. In one aspect, the heterologous Marburg virus transgene encodes a Marburg virus immunogen. In one aspect, the heterologous viral transgene is an Ebola virus transgene. In one aspect, the heterologous Ebola virus transgene encodes an Ebola virus glycoprotein or fragment thereof. In one aspect, the heterologous Ebola virus transgene encodes a Sudan virus immunogen. In one aspect, the heterologous viral transgene is a Sudan virus transgene. In other aspects, the heterologous Sudan virus transgene encodes a Sudan virus glycoprotein or fragment thereof. In one aspect, the heterologous Sudan virus transgene encodes a Sudan virus immunogen.

In other aspects, the one or more sugars comprise trehalose, sucrose, sorbitol, glycerol, mannitol or combinations thereof. In one aspect, the one or more sugars are present in an amount between about 5% to about 40%. In one aspect, the one or more amino acids comprise glutamic acid, histidine, threonine, methionine or glycine. In one aspect, the one or more amino acids comprise histidine, threonine, methionine or glycine. In one aspect, the one or more amino acids are present in an amount between about 50 mM to about 600 mM.

In other aspects, the one or more proteins comprise silk fibroin, albumin, gelatin or combinations thereof. In one aspect, the one or more protein comprises albumin. In one aspect, the one or more protein is not soy peptone. In another aspect, the one or more proteins are present in an amount between about 0.1 g/L to about 15 g/L.

In one aspect, the composition further comprises one or more buffers. In one aspect, the one or more buffers comprise HEPES, a phosphate buffer or combinations thereof. In another aspect, the composition is formulated into a liquid dosage form.

Another embodiment described herein is a pharmaceutical composition comprising: (a) about 5% to about 40% of one or more sugars; (b) about 50 mM to about 600 mM of one or more amino acids; (c) about 0.1 g/L to about 15 g/L of one or more protein; and (d) a virus or viral vector.

In one aspect, the pharmaceutical composition described herein comprises: (a) about 6.10% of one or more sugars; (b) about 288 mM of one or more amino acids; (c) about 9.8 g/L of one or more proteins; and (d) a VSV virus or VSV viral vector. In one aspect, the composition comprises: (a) about 6.10% trehalose; (b) about 141 mM histidine and about 147 mM threonine; (c) about 9.8 g/L of recombinant human serum albumin; (d) about 4.0 g/L potassium phosphate buffer; and about 1×10⁵ to about 1×10⁷ VSV virus or VSV vector viral titer. In one aspect, the VSV virus or VSV viral vector encodes a heterologous LASV viral transgene.

In one aspect, wherein the composition is stable at a temperature less than or equal to at least about 8° C. In one aspect, the viral titer is maintained within 0.5 log titer at 2-8° C. for up to three months. In other aspects, the viral titer is maintained within 0.5 log titer at −20° C. for at least 12 months. In one aspect, the viral titer does not drop by more than about 0.5 log after three months.

In one aspect, the pharmaceutical composition described herein comprises: (a) about 10% of one or more sugars; (b) about 58 mM of one or more amino acids; and (c) the VSV viral vector encodes a heterologous MARV viral transgene. In one aspect, composition comprises a VSV viral titer of about 1×10⁴ to 1×10⁶ TCID₅₀/mL. In another aspect, the composition maintains viral titer after thawing at room temperature for three hours. In one aspect, the composition maintains viral titer at 2-8° C. for up to 30 days.

Another aspect described herein is a method of treating, inhibiting or preventing an infection caused by a virus in a subject in need thereof, comprising administering to the subject an effective amount of the pharmaceutical composition as described herein.

Another aspect described herein is a method of eliciting an immune response in a subject in need thereof, comprising administering to the subject an effective amount of the pharmaceutical composition as described herein.

In one aspect of the methods, the subject is a human or a non-human mammal. In one aspect, the administering is by a route comprising injection. In other aspects, the eliciting an immune response comprises inducing antibody formation. In one aspect, the antibody is an anti-LASV antibody, an anti-MARV antibody, an anti-EBOV antibody or an anti-SUDV antibody.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1: describes the viral genome of the wild-type VSV virus and an exemplary vector as described herein.

FIG. 2: describes the 25° C. stability data of various formulations as part of the initial screening process. Stability of the active ingredient and potential effects of various classes of excipients were studied by individually adding to the drug substance to examine the effects on critical quality attributes.

FIG. 3: describes the prediction profiler model in which amino acids and buffers are the primary factors for stabilizing the rVSV-Lassa antigen at 5° C.

FIG. 4: describes the prediction profiler model in which HSA and cryoprotectant sugars stabilizing frozen rVSV-Lassa antigen at −20° C.

FIG. 5: describes the prediction profiler model in which a combination of all excipients stabilizes the rVSV-Lassa antigen at both 5° C. and −20° C.

FIG. 6: describes results demonstrating that exemplary formulations (1, 2) with HSA had reduced titer loss at both −20° C. and 5° C. for at least 30 days. Formulations (4, 5, 6) with soy peptone had increased titer loss at −20° C. and 5° C. after 30 days with complete loss of infectivity at 25° C. after 30 days at all soy peptone concentration range.

FIG. 7: describes results demonstrating exemplary formulation 1 with 10 g/L HSA outperformed all formulations containing soy peptone and/or lower concentration of HSA at −20° C. after 90 days.

FIG. 8: describes the pH effect on potency (titer) of the EBS-LASV vaccine when evaluated between pH 6.0 to pH 8.0.

FIG. 9: describes the results of freeze-thaw cycles on potency as measured by TCID₅₀.

FIG. 10: describes stability data for the formulation A on EBS-LASV at 2-8° C. and at −20° C.

FIG. 11: describes stability data for the formulation B on EBS-LASV at 2-8° C. and at −20° C.

FIG. 12: describes stability data for formulations C, D and E on EBS-LASV at 2-8° C. and at −20° C.

FIG. 13: describes results of freeze-thaw cycles on potency for formulation A, on EBS-MARV as measured by TCID₅₀.

FIG. 14: describes stability data for formulation A and 2 on EBS-MARV as measured by TCID₅₀.

FIG. 15: describes stability data of EBS-MARV at 25° C., 2-8° C., and −20° C. for Formulations A and 2.

FIG. 16: describes stability data of rVSV-EBOV, rVSV-MARV, and rVSV-SUDV stability at 25° C. for Formulations A, 2, and 3.

FIG. 17 describes stability data of EBS-MARV at 25° C.

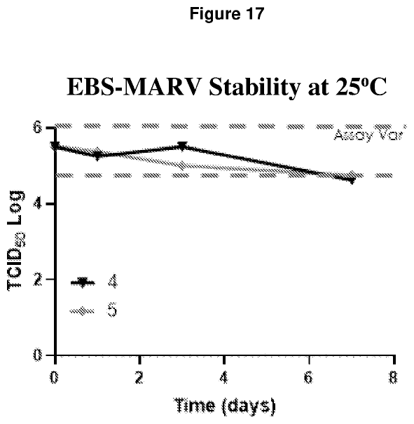

### DETAILED DESCRIPTION OF THE DISCLOSURE

The following description and examples illustrate embodiments of the present disclosure in detail.

It is to be understood that the present disclosure is not limited to the particular embodiments described herein and as such may vary. Those of skill in the art will recognize that there are variations and modifications of the present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure may be described in the context of a single embodiment, the features may also be provided separately or in any suitable combination. Conversely, although the present disclosure may be described herein in the context of separate embodiments for clarity, the present disclosure may also be implemented in a single embodiment. The following definitions supplement those in the art and are directed to the current application and are not to be imputed to any related or unrelated case, e.g., to any commonly owned patent or application. Although any methods and materials similar or equivalent to those described herein may be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, may be used interchangeably. These terms may convey that any combination is specifically contemplated. Solely for illustrative purposes, the following phrases “A, B, and/or C” or “A, B, C, or any combination thereof” may mean “A individually; B individually; C individually; A and B; B and C; A and C; and A, B, and C.” The term “or” may be used conjunctively or disjunctively, unless the context specifically refers to a disjunctive use.

Reference in the specification to “some embodiments,” “some aspects,” “an embodiment,” “an aspect,” “another aspect”, “one embodiment,” “one aspect” or “other embodiments” or “other aspects” means that a particular feature, structure, or characteristic described in connection with the embodiments is included in at least some embodiments, but not necessarily all embodiments, of the present disclosures.

As used in this specification and the claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification may be implemented with respect to any method or composition of the disclosure, and vice versa. Furthermore, compositions of the present disclosure may be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” may mean within 1 or more than 1 standard deviation, per the practice in the art. Alternatively, “about” may mean a range of up to 20%, up to 10%, up to 5%, or up to 1% of a given value. In another example, the amount “about 10” includes 10 and any amounts from 9 to 11. In yet another example, the term “about” in relation to a reference numerical value may also include a range of values plus or minus 10%, 9%, 8%, 7%, 6%, 5%, 4%, 3%, 2%, or 1% from that value. Alternatively, particularly with respect to biological systems or processes, the term “about” may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “bioactivity” of a pharmaceutical composition or vaccine preparation (or of the antigenic or immunogenic components of the vaccine preparation), refers to the ability of the composition or vaccine (or of the antigenic or immunogenic components) to elicit the desired immune response. As a proxy for determining bioactivity of alive and/or attenuated virus composition or vaccine, the titer of the live virus may be measured by any means known in the art, including Tissue Culture Infectious Dose assays (TCID₅₀), which determines the dilution, at which 50% of the virus loaded wells demonstrate a cytopathic effect. The term “potency” as used herein, refers to the titer of a pharmaceutical composition or vaccine preparation, whether measured by infectious units (IU), TCID₅₀, or other methods known in the art.

The term “composition” as used herein is intended to encompass a product comprising specific ingredients in specified amounts, as well as any product which results, directly or indirectly, from combination of the specified ingredients in the specified amounts. By “pharmaceutically acceptable” it is meant the carrier, diluent or excipient must be compatible with the other ingredients of the formulation, including the vectors described herein, and not deleterious to the recipient thereof. A “pharmaceutically acceptable carrier” is any carrier which is relatively non-toxic and innocuous to a patient at concentrations consistent with effective activity of the active ingredient so that any side effects ascribable to the carrier do not vitiate the beneficial effects of the active ingredient.

“Immunogenicity” refers to the ability of a substance, such as an antigen or epitope” to induce a humoral and/or a cell-mediated immunological response in a subject. Immunogenicity may be readily measured by a variety of art-recognized methods.

“Infectivity” as used herein, in reference to a virus means the efficacy of a virus at infecting the cells of a susceptible host and reproducing therein. Any means known in the art may be used to determine virus infectivity as described herein.

“Pathogen” refers to any disease-producing agent that is a virus, bacterium or other microorganism.

“Polynucleotide” or “oligonucleotide” as used herein refers to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs.

Unless otherwise stated, nucleic acid sequences in the text of this specification are given, when read from left to right, in the 5′ to 3′ direction.

The terms “transfection,” “transformation,” “nucleofection,” or “transduction” as used herein refers to the introduction of one or more exogenous polynucleotides into a host cell or organism by using physical, chemical, and/or electrical methods. The nucleic acid sequences and vectors disclosed herein may be introduced into a cell or organism by any such methods, including, for example, by electroporation, calcium phosphate co-precipitation, strontium phosphate DNA co-precipitation, liposome mediated-transfection, DEAE dextran mediated-transfection, polycationic mediated-transfection, tungsten particle-facilitated microparticle bombardment, viral, and/or non-viral mediated transfection. In some cases, the method of introducing nucleic acids into the cell or organism involve the use of viral, retroviral, lentiviral, or transposon, or transposable element-mediated (e.g., Sleeping Beauty) vectors.

“Polypeptide”, “peptide”, and their grammatical equivalents as used herein refer to a polymer of amino acid residues. The polypeptide may optionally include glycosylation or other modifications typical for a given protein in a given cellular environment. Polypeptides and proteins disclosed herein (including functional portions and functional variants thereof) may comprise synthetic amino acids in place of one or more naturally-occurring amino acids. Such synthetic amino acids are known in the art, and include, for example, aminocyclohexane carboxylic acid, norleucine, α-amino n-demayoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides or proteins described herein in an engineered cell may be associated with post-translational modifications of one or more amino acids of the polypeptide or protein. Non-limiting examples of post-translational modifications include phosphorylation, acylation including acetylation and formylation, glycosylation (including N-linked and O-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

An “expression vector” or “vector” is any nucleic acid molecule or genetic element (e.g., a plasmid, a mini-circle, a nanoplasmid, chromosome, virus, transposon) containing a gene or specific nucleic acid sequence. An expression vector is typically used to introduce a specific nucleic acid into a (target) cell for expression of the nucleic acid by the cell, e.g., to produce one or more proteins or mRNAs encoded by the nucleic acid, e.g., by a constitutive or an inducible promoter. A vector or expression vector are typically expressed in a host cell, and, furthermore, contains the necessary elements to control expression of the gene. Typically, an expression vector comprises a transcription promoter, a gene of interest, and a transcription terminator. Suitable vectors include, but are not limited to, plasmids, transposons, bacteriophages, cosmids or virus based vectors. Vectors may contain polynucleotide sequences which are able to effect ligation, recombination or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Expression vectors may be capable of directly expressing nucleic acid sequence products encoded therein without ligation or integration of the vector into host cell DNA sequences. In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11:1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP.

The term “operably linked” as used herein refers to the physical and/or functional linkage of a DNA segment to another DNA segment in such a way as to allow the segments to function in their intended manners. A DNA sequence encoding a gene product is operably linked to a regulatory sequence when it is linked to the regulatory sequence, such as, for example, promoters, enhancers and/or silencers, in a manner, which allows modulation of transcription of the DNA sequence, directly or indirectly. For example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. An enhancer or silencer is operably linked to a DNA sequence coding for a gene product when it is ligated to the DNA sequence in such a manner as to increase or decrease, respectively, the transcription of the DNA sequence. Enhancers and silencers may be located upstream, downstream or embedded within the coding regions of the DNA sequence. A DNA for a signal sequence is operably linked to DNA coding for a polypeptide if the signal sequence is expressed as a pre-protein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

The term “induce”, “induction” and its grammatical equivalents as used herein may refer to an increase in nucleic acid sequence transcription, promoter activity and/or expression brought about by a transcriptional regulator, relative to some basal level of transcription. “Induce” may also refer to the generating of an immune response.

“Patient” or “subject” as used herein refers to a mammalian subject Exemplary patients may be humans, apes, dogs, pigs, cattle, cats, horses, goats, sheep, rodents and other mammalians that may benefit from the therapies disclosed herein. Exemplary human patients may be male and/or female. “Patient in need thereof” or “subject in need thereof” is referred to herein as a patient that may benefit from a prophylactic treatment, such as a vaccine, or diagnosed with or suspected of having a disease or disorder.

“Administering” is referred to herein as providing one or more compositions described herein to a patient or a subject, e.g., a VSV virus or vector. By way of example and not limitation, composition administration, e.g., injection, may be performed by intravenous (i.v.) injection, sub-cutaneous (s.c.) injection, intradermal (i.d.) injection, intraperitoneal (i.p.) injection, or intramuscular (i.m.) injection. One or more such routes may be employed. Parenteral administration may be, for example, by bolus injection or by gradual perfusion over time. Alternatively, or concurrently, administration may be by the oral route. In an embodiment, a composition of the present disclosure may comprise a vector comprising a nucleic acid sequence described herein, in an amount that is effective to treat, inhibit or prevent infection. A pharmaceutical composition may comprise a vector as described herein, in combination with one or more pharmaceutically or physiologically acceptable carriers, diluents or excipients.

As used herein, the term “stabilizing”, “stabilize,” “stability,” and “stabilization,” refer to the retaining of bioactivity, e.g., of at least one antigen in a vaccine preparation (e.g., immunogenicity, antibody response, and T-cell response), or one virus (e.g., infectivity and infectious particle titer) in a viral preparation, such that for example, one or more antigens or viruses in a formulation retain at least about 30% of its original bioactivity, at least about 40%, at least about 50%, at least about 60%, at least about 70%, at least about 80%, or at least about 90% of its original bioactivity. Furthermore, in the context of the compositions described herein, “stable” may also refer to a change in TCID₅₀ log titer of the VSV virus or rVSV vector over time, such that a composition that exhibits a log titer loss of less than or equal to 1 log may be considered stable.

As used herein, the term “treatment”, “treating”, or its grammatical equivalents refers to obtaining a desired pharmacologic and/or physiologic effect. In some embodiments, the effect is therapeutic, i.e., the effect partially or completely cures a disease and/or adverse symptom attributable to the disease. In some embodiments, the term “treating” may include inhibiting or preventing a disease or a condition, including infection.

As used herein, a “treatment interval” refers to a treatment cycle, for example, a course of administration of a therapeutic agent that may be repeated, e.g., on a regular schedule. In some embodiments, a dosage regimen may have one or more periods of no administration of the therapeutic agent in between treatment intervals.

The terms “administered in combination” or “co-administration” or “co-administering” or “co-providing” as used herein, means that two (or more) different treatments are delivered to the subject during the course of or prior to the subject's affliction with a disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments may be partially additive, wholly additive, or greater than additive. The delivery may be such that an effect of the first treatment delivered is still detectable when the second is delivered.

In some embodiments, the first treatment and second treatment may be administered simultaneously (e.g., at the same time), in the same or in separate compositions, or sequentially. Sequential administration refers to administration of one treatment before (e.g., immediately before, less than 5, 10, 15, 30, 45, 60 minutes; 1, 2, 3, 4, 6, 8, 10, 12, 16, 20, 24, 48, 72, 96 or more hours; 4, 5, 6, 7, 8, 9 or more days; 1, 2, 3, 4, 5, 6, 7, 8 or more weeks before) administration of an additional, e.g., secondary, treatment. The order of administration of the first and secondary treatment may also be reversed.

The term n “therapeutically effective amount”, “therapeutic amount”, “immunologically effective amount”, or its grammatical equivalents refers to an amount effective, at dosages and for periods of time necessary, to achieve a desired therapeutic result. The therapeutically effective amount may vary according to factors such as the disease state, age, sex, and weight of the individual, and the ability of a composition described herein to elicit a desired response in one or more subjects. The precise amount of the compositions of the present disclosure to be administered may be determined by a physician with consideration of individual differences in age, weight, tumor size, extent of inflammation, infection or metastasis, and condition of the patient (subject).

The term “vaccine” as used herein refers to any preparation of an antigen (including subunit antigens, toxoid antigens, conjugate antigens, or other types of antigen molecules) or a killed or live attenuated microorganism that, when introduced into a subject's body, affects the immune response to the specific antigen or microorganism by causing activation of the immune system against the specific antigen or microorganism, including, for example, inducing antibody formation, T cell responses, and/or B-cell responses. In some embodiments, a vaccine is a VSV vector expressing an antigen or protein from an infectious agent, wherein the vaccine induces an immune response in a subject that inhibits or would inhibit the infectious agent.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The present disclosure describes a novel composition comprising a virus or a viral vector and methods of treatment using the same. Thus, one embodiment described herein is a pharmaceutical composition comprising a virus or viral vector. Another embodiment is a viral vector for the expression of Lassa virus (LASV) glycoproteins and methods of treatment using the same.

Lassa virus (LASV) is an enveloped virus in the family Arenaviridae. It harbors a bi-segmented single-stranded ambisense RNA genome, with each segment encoding two proteins. The large segment (˜7.3 kilobases (kb)) encoding a zinc-binding matrix protein (Z) and the RNA-dependent RNA polymerase (L); the small (˜3.5 kb) segment encodes the nucleoprotein (NP), and the glycoprotein precursor complex (GPC). LASV glycoprotein may be used to induce a host-derived immune response against LASV. Also described herein are novel vectors for the delivery of the LASV glycoprotein, including recombinant Vesicular Stomatitis Virus (rVSV) vector platforms. Similarly, Marburg virus (MARV) and the ebolaviruses are filamentous, enveloped, negative-sense, single-stranded RNA (ssRNA) viruses that comprise a 19.1 kb non-segmented, single-stranded, negative-sense RNA genome, with structural domains that include an N-terminal arm, an NP core composed of N- and C-terminal lobes, a disordered linker, and a C-terminal tail.

Due to the natural instability of live virus, many current commercial and emergency vaccines containing live virus are manufactured as liquid vaccines containing live virus, where storage durations are below freezing, e.g., −80° C. The vectors of the present disclosure may be utilized to achieve the desired pharmacological effect by administration to a patient in need thereof in an appropriately formulated pharmaceutical composition. The present disclosure describes a substantially liquid vaccine preparation with surprisingly increased stability over time and/or at elevated temperatures. Thus, appropriate pharmaceutical compositions comprising the vectors are contemplated herein.

One embodiment described herein is a liquid pharmaceutical composition comprising one or more vectors or viruses described herein. In one aspect, the pharmaceutical composition comprises that shown in Table 1.

TABLE 1
Exemplary Compositions

| Exemplary Ingredients | Amount | |
|---|---|---|
| EBS-LASV(rVSVn4Δg-LASVgpc1) | TCID₅₀/mL = 1 × 10² − 1 × 10¹⁰ | |
| Sugar(s) | 5-40% | |
| Amino Acid(s) | 50-600 | mM |
| Protein(s) | 0.1-15 | g/L |
| Buffer(s) | 2-25 | mM |

It is understood that while Table 1 describes the formulation using a LASV transgene, the same formulations may be formulated using any of the viral transgenes described herein, including the MARV transgene, the EBOV transgene, or the SUDV transgene or with a VSV virus containing no heterologous transgene and/or containing genetic manipulation(s) of the VSV genome.

Thus, in one aspect, the composition comprises an rVSV vector or virus as the active ingredient, one or more sugars, one or more amino acids, one or more proteins and one or more buffers.

Viruses themselves, whether mutated, genetically engineered or wild-type, are useful in research or therapeutic treatments and with the formulations described herein. For example, stocks of viruses are stored for research purposes, e.g., to test anti-viral drugs and in viral research. Also, viruses are used as therapeutics, such oncolytic viruses, in which viral stocks are needed.

Viral vectors may be used as delivery vehicles for nucleic acids encoding therapeutic molecules and provide a suitable delivery and expression vehicle for exogenous proteins. Retrovirus, including lentivirus, adenovirus, adeno-associated virus (AAV) and herpes simplex virus have all been adapted for viral vector applications (Robbins et al., 1998). The term “viral vector” as used herein may thus refer either to a virus or viral particle capable of transferring a nucleic acid into a cell or to the transferred nucleic acid itself. Any type of virus may be used as a viral vector.

Rhabdovirus based vectors are of particular interest. The Rhabdoviridae family are single-stranded RNA viruses and have been used as the basis for vaccine platforms, and in some cases the Rhadbovirus is attenuated. (Zemp et al., 2018). These viruses have a small, single segment viral genome that encodes five proteins: the nucleoprotein (N), phosphoprotein (P), matrix protein (M), glycoprotein (G), and RNA dependent RNA polymerase (L), which are manipulatable, allowing for optimized expression. A variety of manipulations have been employed with Rhabdovirus to attenuate the virus, including modulating viral replication to a single cycle; reducing expression of viral genes by N gene translocation; truncation of G protein to impact maturation of the virus; and engineering mutations in the M protein. (Publicover, Ramsburg, & Rose, 2005; Ball, Pringle, Flanagan, Perepelitsa, & Wertz, 1999; Wertz, Perepelitsa, & Ball, 1998). Thus, in one aspect, the virus described herein is a Rhabodovirus or a Rhabdovirus based vector.

Several Rhabdovirus family members are currently being used in the development of viral vector vaccines, including Rabies virus (RABV), Vesicular Stomatitis Virus (VSV) and recombinant Maraba Virus. Recombinant RABV vectors such as rHEP5.0-CVSG are engineered vectors where the VSV G gene is deleted. (Ohara et al., 2013). Maraba viral vectors has been used for cancer immunotherapy because of their oncolytic activity and ability to elicit a robust immune response. (Pol et al., 2014).

One member of the Rhabdoviridae family is the Vesicular Stomatitis Virus (VSV) from the Vesiculoviridae genus. Several VSV-based vaccines are currently in development for various uses, including Ebola, and human immunodeficiency virus (HIV-1). A highly attenuated vector known as rVSVn4CT1gag1 was developed as an HIV-1 candidate vaccine and reportedly demonstrated safety and immunogenicity in a several clinical trials (study HVTN 090, ClinicalTrials.gov NCT01438606 and study HVTN 087, ClinicalTrials.gov NCT01578889). Ebola vaccine vector rVSVN4CT1-EBOVGP1 was reportedly well tolerated at all dose levels tested and was immunogenic despite a high degree of attenuation in a Phase 1 trial (study rVSV-EBOV-01, ClinicalTrials.gov NCT02718469).

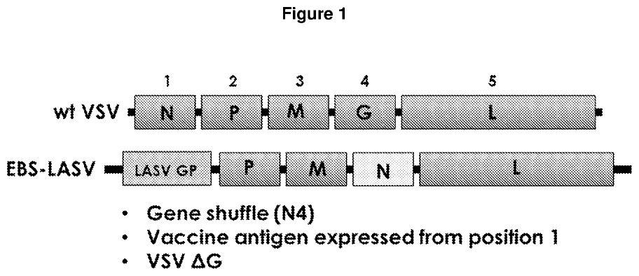

FIG. 1 describes the viral genome of a wild-type VSV. Vectors using VSV may be based on genetic modifications that achieve attenuation of the virus' pathogenicity. These genetic modifications include deletions, mutations, gene shuffling, or truncation of various viral proteins. For example, one modification includes the deletion of the VSV glycoprotein gene and, in some cases, replacement by insertion of any antigen of interest in its place. Since the wild-type VSV glycoprotein is responsible for pathogenicity, deletion of this gene contributes to attenuation, an important consideration given these vectors remain replication competent. (Fathi et al., 2019). Thus, in one aspect, the viral vector described herein is a Vesicular Stomatitis Virus (VSV) vector.

Recombinant VSV (rVSV) vectors may be designed using a variety of attenuation strategies involving engineering of the viral genome. For example, translocation of the N protein gene from its wild-type 3′ promoter-proximal location at the 1^{st} position to any downstream position results in reduced viral replication. In another example, the N gene may be “shuffled” between the P and M protein genes, between the M and G protein genes or between the G or L protein genes. Thus, the N gene may be shuffled to any non-wild-type location. Thus, in one aspect the vector of the instant disclosure comprises a mutated viral genome. In some aspects, the mutated genome comprises a 3′-PNMGL-5′, a 3′-PMNGL-5′, a 3′-PNMXL-5′ or 3′-XPMNL-5′ arrangement of viral genes relative to the wild-type genome, wherein N is the nucleocapsid protein gene, P is the phosphoprotein gene, M is the matrix protein gene, G is the glycoprotein gene and X may be an antigen gene of interest.

Any transgene may be inserted into the viral vectors of the instant disclosure depending on the use of the vector, e.g., insertion of a transgene encoding an antigen, therapeutic protein or reporter gene. These heterologous transgene sequences include but are not limited to a HIV gene, a HTLV gene, a SIV gene, a RSV gene, a PIV gene, a HSV gene, a CMV gene, an Epstein-Barr virus gene, a Varicella-Zoster virus gene, a mumps virus gene, a measles virus gene, an influenza virus gene, a poliovirus gene, a rhinovirus gene, a hepatitis A virus gene, a hepatitis B virus gene, a hepatitis C virus gene, a Norwalk virus gene, a togavirus gene, an alphavirus gene, a rubella virus gene, a rabies virus gene, a Marburg virus gene, an Ebola virus gene, a Lassa virus gene, a Sudan virus gene, a papilloma virus gene, a polyoma virus gene, a metapneumovirus gene, a coronavirus gene, a Vibrio cholera gene, a Streptococcus pneumoniae gene, a Streptococcus pyogenes gene, a Streptococcus agalactiae gene, a Neisseria meningitidis gene, a Neisseria gonorrheae gene, a Corynebacteria diphtheria gene, a Clostridium tetani gene, a Bordetella pertussis gene, a Helicobacter pylori gene, a Haemophilus gene, a Chlamydia gene, a Escherichia coli gene, a cytokine gene, a T-helper epitope, a CTL epitope, an adjuvant gene or a co-factor gene.

Other examples of rVSV genetic modifications involve truncation of the cytoplasmic tail of the G protein to impair viral core protein interactions, and impede viral maturation. Mutations or deletion in the M protein may be made to reduce virulence. Any attenuation strategy may be used with the vectors described herein, alone or in combination. In some cases, the use of multiple modes of attenuation may in some case offer a synergistic effect in muting the host innate immune response.

FIG. 1 describes one aspect of the vector of the present disclosure, in which the VSV G gene is deleted, the N gene is translocated or “shuffled” to the 4th position between the M and L protein genes, and a Lassa glycoprotein gene is inserted into the 1^{st} position in place of the location of N gene in the wild-type virus. Thus in one aspect, the vector encodes a heterologous viral protein or fragment thereof. In yet another aspect, the viral vector encodes a heterologous immunogen. In one aspect, the heterologous viral protein is a Lassa virus glycoprotein. In some aspects, the vector comprises a Lassa virus glycoprotein at the 1^{st} position of the viral genome. In some aspects, the vector does not encode a homologous viral glycoprotein or fragment thereof, such as VSV G protein. In some aspects, the vector comprises an N gene at the 4th position of the viral genome. In some aspects, the vector comprises both the VSV G gene and a heterologous viral transgene, for examples a Lassa virus glycoprotein transgene, a Marburg virus glycoprotein transgene, an Ebola virus glycoprotein transgene, or a Sudan virus glycoprotein transgene. In other aspects, the VSV G gene is deleted, and the vector includes a heterologous viral transgene.

Thus one aspect described herein is a pharmaceutical composition comprising: a Lassa virus immunogen; one or more sugars; one or more amino acids; one or more proteins; and one or more buffers. In other aspects, the pharmaceutical composition comprises a Marburg virus immunogen, one or more sugars; one or more amino acids; one or more proteins; and one or more buffers. In other aspects, the pharmaceutical composition comprises a Ebola virus immunogen, one or more sugars; one or more amino acids; one or more proteins; and one or more buffers. In one aspect, the pharmaceutical composition comprises a Sudan virus immunogen, one or more sugars; one or more amino acids; one or more proteins; and one or more buffers.

The active ingredient may be a VSV virus or any rVSV vector, e.g., as described herein. Appropriate virus titers may be determined by any means in the art, including Tissue Culture Infectious Dose (TCID₅₀) assays, which determines the dilution, at which 50% of the virus-loaded wells demonstrate a cytopathic effect. Thus, in one aspect, the rVSV vector is present in the pharmaceutical composition in any amount between about 1×10²-1×10¹⁰ as measured by TCID₅₀/mL. In another aspect, the rVSV vector is present in any amount between about 1×10³-1×10⁹ TCID₅₀/mL. In some aspects, the rVSV vector is present in any amount between about 1×10⁴-1×10⁸ TCID₅₀/mL. In one aspect, the rVSV vector is present in any amount between about 1×10⁵-1×10⁷ TCID₅₀/mL. In one aspect, the rVSV vector is present in an amount of about 1×10² TCID₅₀/mL. In some aspects, the rVSV vector is present in an amount of about 1×10³. In another aspect, the rVSV vector is present in an amount of about 1×10⁴ TCID₅₀/mL. In one aspect, the rVSV vector is present in an amount of about 1×10⁵ TCID₅₀/mL. In some aspects, the rVSV vector is present in an amount of about 1×10⁶ TCID₅₀/mL. In some aspects, the rVSV vector is present in an amount of about 1×10⁷ TCID₅₀/mL. In one aspect, the rVSV vector is present in an amount of about 1×10⁸ TCID₅₀/mL. In another aspect, the rVSV vector is present in an amount of about 1×10⁹ TCID₅₀/mL.

Sugars are known protein stabilizers and thus considered a promising excipient for virus or viral vector based vaccines. Various sugars may be used in the pharmaceutical compositions described herein, including one or more of sucrose, lactose, sorbitol, trehalose, mannitol, D-mannose, D-fructose, dextrose, glycerin, or combinations thereof. In one aspect, the one or more sugar of the pharmaceutical composition described herein comprises trehalose, sucrose, sorbitol, glycerol, mannitol or a combination thereof.

In some aspects, the one or more sugar may be present in the pharmaceutical composition described herein in any amount between about 5% to about 40%. In another aspect, the one or more sugar may be present in an amount between about 6% to about 30%. In another aspect, the one or more sugar may be present in an amount between about 7% to about 20%. In yet another aspect, the one or more sugar may be present in an amount between about 8% to about 10%, 5% to about 7%, 5.5% to about 6.5%, 10% to about 15%, 11% to about 14%, 12% to about 13%, 20% to about 28%, 22% to about 26%, 24% to about 25%, 25% to about 35%, 26% to about 34%, 27% to about 33%, 28% to about 32%, or 29.5% to about 31.5%. In another aspect, the one or more sugar may be present in an amount of about 5%. In one aspect, the one or more sugar may be present in an amount of about 6%. In yet another aspect, the one or more sugar may be present in an amount of about 7%. In one aspect, the one or more sugar may be present in an amount of about 8%. In another aspect, the one or more sugar may be present in an amount of about 9%. In yet another aspect, the one or more sugar may be present in an amount of about 10%. In one aspect, the one or more sugar may be present in an amount of about 11%. In another aspect, the one or more sugar may be present in an amount of about 12%. In yet another aspect, the one or more sugar may be present in an amount of about 13%. In one aspect, the one or more sugar may be present in an amount of about 14%. In another aspect, the one or more sugar may be present in an amount of about 15%. In yet another aspect, the one or more sugar may be present in an amount of about 16%. In one aspect, the one or more sugar may be present in an amount of about 17%. In yet another aspect, the one or more sugar may be present in an amount of about 18%. In one aspect, the one or more sugar may be present in an amount of about 19%. In another aspect, the one or more sugar may be present in an amount of about 20%. In one aspect, the one or more sugar may be present in an amount of about 21%. In another aspect, the one or more sugar may be present in an amount of about 22%. In another aspect, the one or more sugar may be present in an amount of about 23%. In yet another aspect, the one or more sugar may be present in an amount of about 24%. In one aspect, the one or more sugar may be present in an amount of about 25%. In another aspect, the one or more sugar may be present in an amount of about 26%. In yet another aspect, the one or more sugar may be present in an amount of about 27%. In one aspect, the one or more sugar may be present in an amount of about 28%. In another aspect, the one or more sugar may be present in an amount of about 29%. In yet another aspect, the one or more sugar may be present in an amount of about 30%.

In some aspects, a composition comprises sucrose, but does not comprise or does not contain a substantial or effective amount of mannitol. In some aspects, a composition comprises sucrose, but does not comprise or does not contain a substantial or effective amount of trehalose. In some aspects, a composition comprises sucrose, but does not comprise or does not contain a substantial or effective amount of another sugar.

In some aspects, a composition comprises sucrose and trehalose, but does not comprise or does not contain a substantial or effective amount of mannitol. In some aspects, a composition comprises sucrose and mannitol, but does not comprise or does not contain a substantial or effective amount of trehalose. In some aspects, a composition comprises sucrose and trehalose, but does not comprise or does not contain a substantial or effective amount of another sugar. In some aspects, a composition comprises sucrose and mannitol, but does not comprise or does not contain a substantial or effective amount of another sugar.

In some aspects, a composition does not comprise or does not contain a substantial or effective amount of sucrose. In some aspects, a composition comprises no more than 0.05, 0.1, 0.2, 0.3, 0.4, 0.5, 0.6, 0.7, 0.8, 0.9, or 1% sucrose. In some aspects, a composition does not comprise, or does not contain a substantial or effective amount of, one or more of a-cyclodextrin, dextran, or sorbitol.

In some aspects, trehalose may be present in the pharmaceutical composition described herein in any amount between about 4% to about 8%, about 5% to about 7%, about 6% to about 6.5%, about 6.1%, about 6.4%, about 5% to about 13%, about 6 to about 12.5%, about 12.3%, or about 12.4%. In some aspects, the pharmaceutical composition does not comprises trehalose or does not comprise more than 0.2% trehalose.

In some aspects, sucrose may be present in the pharmaceutical composition described herein in any amount between about 0.05% to about 0.3%, about 0.1% to about 0.2%, about 0.15%, about 10.3% to about 14.5%, about 11.8% to about 13.2%, about 11.5% to about 12.5%, about 12.4%. In some aspects, the pharmaceutical composition does not comprise any or no more than about 0.09% to about 0.3%, about 0.1% to about 0.2%, about 0.15% of sucrose. In some aspects, the pharmaceutical composition does not comprises sucrose.

In some aspects, mannitol may be present in the pharmaceutical composition described herein in any amount between about 8.1% to about 15.6%, about 9.5% to about 14.3%, about 10.5% to about 13%, about 12.2%, about 12.3%, about 12.4%, or about 12.5%. In some aspects the pharmaceutical composition does not comprise mannitol.

In some aspects, the pharmaceutical composition described herein comprises trehalose, sucrose or the combined concentrations or trehalose and sucrose together in an amount between about 1.2% to about 19.8%, about 3.5% to 18.8%, about 4.5% to about 15.4%, about 5.5% to about 13.4%, about 6.10%, about 6.3%, about 8.2%, about 10.1%, about 12.30%, about 14.7%, about 16.3%, or about 18.5%.

In some aspects, the pharmaceutical composition described herein comprises sucrose, mannitol or the combined concentrations or sucrose and mannitol together in an amount between about 7.5% to about 30.1%, about 10.3% to about 28.7%, about 11.5% to about 26.4%, about 12% to about 24.1%, about 14.3% to about 22.6%, about 8.9%, about 10.6%, about 12.3%, about 16.3%, about 19.7%, about 21.5%, about 24.7%, or about 27.8%

In some aspects, the pharmaceutical composition comprises trehalose, mannitol or the combined concentrations of trehalose and mannitol together in any amount between about 5.3% to about 31.5%, about 8.6% to about 27.2%, about 10.9% to about 24.6%, about 15% to about 20%, about 17% to about 19%, about 18% to about 19%, about 20% to about 27.5%, about 22.2% to about 25.5%, about 23.5% to about 25%, about 9.3%, about 12.30%, about 15.7%, about 18.4%, about 21.8%, about 24.6%, about 27.7%, or about 30.8%.

In some aspects, the pharmaceutical composition comprises a total sugar amount of between about 4% to about 8%, about 5% to about 7%, about 2% to about 14%, about 13% to about 13%, about 1% to about 40%, about 2% to about 35%, about 5% to about 30%, about 5.5% to about 20%, about 10.7% to about 25.7%, about 5.5% to about 7.5%, about 6% to about 7%, about 20.2% to about 25.6%, about 23.1% to about 25.4%, about 24% to about 25%, about 25.9% to about 32.4%, about 30% to about 31%, about 38% to about 31.7%, about 6%, about 6.1%, about 6.5%, about 12.3%, about 24.1%, about 24.6%, about 28.1%, about 30.8%, or about 31%.

In some aspects, the pharmaceutical composition comprises mannitol, sucrose and/or trehalose, but does not comprise, or does not contain a substantial or effective amount of another sugar.

Amino acids for use in the pharmaceutical compositions described herein may include one or more of glycine, alanine, asparagine, aspartic acid, glutamic acid, histidine, leucine, lysine, methionine, phenylalanine, proline, serine, threonine, valine or combinations thereof. Amino acids may include the L or D stereoisomer. In one aspect, the one or more amino acids in the pharmaceutical composition described herein comprises glutamic acid, histidine, threonine, methionine, glycine or combinations thereof.

In some aspects, the one or more amino acids in the pharmaceutical composition described herein comprises histidine, citrulline, serine, glycine, and/or valine. In some aspects, the pharmaceutical composition comprises (i) histidine and threonine, (ii) histidine and glycine, or (iii) histidine, threonine, methionine and glycine. In some aspects, the pharmaceutical composition comprises (i) histidine and threonine, but does not comprise methionine and glycine or (ii) histidine and glycine, but does not comprise threonine and methionine. In some aspects, the pharmaceutical composition does not comprise glutamic acid or any other amino acids. In some aspects, the pharmaceutical composition comprises no more than 0.01, 0.02, 0.03, 0.04, 0.05, 0.06, 0.07. 0.08, 0.09, or 1.0 grams/liter of glutamic acid.

In some aspects, the pharmaceutical composition comprises (i) histidine and threonine, but does not comprise another amino acid excipient, (ii) histidine and glycine, but does not comprise another amino acid excipient, or (iii) histidine, threonine, methionine and glycine, but does not comprise another amino acid excipient. In some aspects, the pharmaceutical composition comprises (i) glutamic acid, histidine and threonine, (ii) glutamic acid, histidine and glycine, or (iii) glutamic acid, histidine, threonine, methionine and glycine. In some aspects, the pharmaceutical composition comprises (i) glutamic acid, histidine and threonine, but does not comprise methionine and glycine or (ii) glutamic acid, histidine and glycine, but does not comprise threonine and methionine. In some aspects, the pharmaceutical composition comprises (i) glutamic acid, histidine and threonine, but does not comprise another amino acid excipient (ii) glutamic acid, histidine and glycine, but does not comprise another amino acid excipient, or (iii) glutamic acid, histidine, threonine, methionine and glycine, but does not comprise another amino acid excipient.

In some aspects, the composition does not comprise one or more or does not contain a substantial or effective amount of the following excipients: methionine, glutathione, glycine, arginine, lysine, alanine, proline, cysteine, tryptophan, serine, leucine, valine, isoleucine, phenylalanine or threonine. In some aspects, a composition does not comprise, or does not contain a substantial or effective amount of, one or more of phenylalanine, lysine, arginine, cysteine, aspartic acid, glutamic acid, leucine, or tryptophan.

In some aspects, the one or more amino acids may be present in the pharmaceutical composition described herein in any amount between about 50 mM to about 600 mM. In another aspect, the one or more amino acids may be present in any amount between about 60 mM to about 500 mM. In yet another aspect, the one or more amino acids may be present in any amount between about 70 mM to about 400 mM. In another aspect, the one or more amino acids may be present in any amount between about 80 mM to about 300 mM. In yet another aspect, the one or more amino acids may be present in any amount between about 90 mM to about 400 mM. In another aspect, the one or more amino acids may be present in any amount between about 100 mM to about 300 mM. In one aspect, the one or more amino acids may be present in an amount of about 50 mM.

In some aspects, histidine may be present in the pharmaceutical composition described herein in any amount between about 100 mM to about 175 mM, about 120 mM to about 160 mM, about 130 mM to about 150 mM, about 140.1 mM to about 148.8 mM, about 145.1 mM to about 149.2 mM, about 142.4 mM to about 148.1 mM, about 141 mM, about 143.5 mM, about 145.7 mM, about 147 mM or about 149.1 mM. In some aspects, the pharmaceutical composition does not comprise histidine. In some aspects, the pharmaceutical composition comprises histidine and does not comprise another amino acid or a substantial or effective amount of another amino acid.

In some aspects, threonine may be present in the pharmaceutical composition described herein in any amount between about 50 mM to about 175 mM, about 60 mM to about 160 mM, about 70 mM to about 150 mM, about 72.5 mM to about 148.1 mM, about 70 mM to about 75 mM, about 72 mM to about 75 mM, about 73 mM to about 74 mM, about 75.3 mM to about 149.1 mM, about 80.2 mM to about 148.6 mM, about 70.3 mM, about 145 mM to about 150 mM, about 146 mM to about 148 mM, about 146.5 mM to about 147.5 mM, about 73.5 mM, about 145.1 mM, about 147 mM or about 149.8 mM. In some aspects, the pharmaceutical composition does not comprise threonine. In some aspects, the pharmaceutical composition comprises threonine and does not comprise another amino acid or a substantial or effective amount of another amino acid.

In some aspects, methionine may be present in the pharmaceutical composition described herein in any amount between about 52 mM to about 174 mM, about 65 mM to about 167 mM, about 71 mM to about 155 mM, about 72 mM to about 148 mM, about 75.1 mM to about 149.3 mM, about 80.5 mM to about 148.9 mM, about 145 mM to about 150 mM, about 146 mM to about 148 mM, about 146.5 mM to about 147.5 mM, about 70.4 mM, about 73.5 mM, about 145 mM, about 147.2 mM or about 149 mM. In some aspects, the pharmaceutical composition does not comprise methionine. In some aspects, the pharmaceutical composition comprises methionine and does not comprise another amino acid or a substantial or effective amount of another amino acid.

In some aspects, glycine may be present in the pharmaceutical composition described herein in any amount between about 50.8 mM to about 175.4 mM, about 60 mM to about 160.1 mM, about 70.3 mM to about 155 mM, about 72.1 mM to about 148.5 mM, about 75 mM to about 149 mM, about 82 mM to about 148.4 mM, about 145 mM to about 150 mM, about 146 mM to about 148 mM, about 146.5 mM to about 147.5 mM, about 70 mM, about 73.5 mM, about 145.7 mM, about 147 mM or about 149.1 mM. In some aspects, the pharmaceutical composition does not comprise glycine. In some aspects, the pharmaceutical composition comprises glycine and does not comprise another amino acid or a substantial or effective amount of another amino acid.

In some aspects, the pharmaceutical composition herein comprises histidine and/or threonine, each in any amount between about 52 mM to about 174 mM, about 60 mM to about 160 mM, about 70 mM to about 150 mM, about 72.1 mM to about 148.4 mM, about 75.9 mM to about 149.1 mM, about 80.3 mM to about 148.8 mM, about 145 mM to about 150 mM, about 146 mM to about 148 mM, about 146.5 mM to about 147.5 mM, about 70.2 mM, about 73.5 mM, about 145.4 mM, about 147 mM or about 148 mM. In some aspects, the pharmaceutical composition comprises histidine and threonine, and does not comprise another amino acid or a substantial or effective amount of another amino acid.

In some aspects, the pharmaceutical composition comprises histidine and/or methionine, each in any amount between about 102 mM to about 176 mM, about 123 mM to about 167 mM, about 134 mM to about 151 mM, about 147 mM to about 149.7 mM, about 141.5 mM to about 149.1 mM, about 142.3 mM to about 148.4 mM, about 145 mM to about 150 mM, about 146 mM to about 148 mM, about 146.5 mM to about 147.5 mM, about 141.2 mM, about 143.5 mM, about 145.9 mM, about 147 mM or about 149.1 mM.

In some aspects, the pharmaceutical composition comprises histidine and/or glycine, each in any amount between about 100 mM to about 175 mM, about 120 mM to about 160 mM, about 130 mM to about 150 mM, about 140 mM to about 148 mM, about 141.4 mM to about 149.2 mM, about 142.7 mM to about 148.1 mM, about 145 mM to about 150 mM, about 146 mM to about 148 mM, about 146.5 mM to about 147.5 mM, about 141 mM, about 143.7 mM, about 145.4 mM, about 147 mM or about 149 mM. In some aspects, the pharmaceutical composition comprises histidine and glycine, and does not comprise another amino acid or a substantial or effective amount of another amino acid.

In some aspects, the pharmaceutical composition comprises threonine and/or methionine, each in any amount between about 50 mM to about 175 mM, about 60 mM to about 160 mM, about 70 mM to about 150 mM, about 72 mM to about 148 mM, about 75.7 mM to about 149.4 mM, about 80.5 mM to about 148.1 mM, about 70 mM to about 75 mM, about 72 mM to about 75 mM, about 73 mM to about 74 mM, about 145 mM to about 150 mM, about 146 mM to about 148 mM, about 146.5 mM to about 147.5 mM, about 70.2 mM, about 73.5 mM, about 144.6 mM, about 147 mM or about 148.4 mM. In some aspects, the pharmaceutical composition comprises methionine and threonine, and does not comprise another amino acid or a substantial or effective amount of another amino acid.

In some aspects, the pharmaceutical composition comprises threonine and/or glycine, each in any amount between about 50 mM to about 175 mM, about 60 mM to about 160 mM, about 70 mM to about 150 mM, about 72 mM to about 148 mM, about 75 mM to about 149 mM, about 80 mM to about 148 mM, about 70 mM to about 75 mM, about 72 mM to about 75 mM, about 73 mM to about 74 mM, about 145 mM to about 150 mM, about 146 mM to about 148 mM, about 146.5 mM to about 147.5 mM, about 70 mM, about 73.5 mM, about 145 mM, about 147 mM or about 149 mM. In some aspects, the pharmaceutical composition comprises glycine and threonine, and does not comprise another amino acid or a substantial or effective amount of another amino acid.

In some aspects, the pharmaceutical composition comprises methionine and/or glycine, each in any amount between about 100.1 mM to about 175.5 mM, about 120.2 mM to about 160.3 mM, about 130.4 mM to about 150.5 mM, about 140.6 mM to about 148.7 mM, about 141.9 mM to about 149 mM, about 142.1 mM to about 148.2 mM, about 145 mM to about 150 mM, about 146 mM to about 148 mM, about 146.5 mM to about 147.5 mM, about 141.4 mM, about 143 mM, about 145.5 mM, about 147 mM or about 148.6 mM. In some aspects, the pharmaceutical composition comprises methionine and glycine, and does not comprise another amino acid or a substantial or effective amount of another amino acid.

In some aspects, the pharmaceutical composition comprises histidine, methionine, glycine and threonine, and does not comprise another amino acid or a substantial or effective amount of another amino acid.

In some aspects, the pharmaceutical composition comprises a total amino acid concentration of about 250 mM to about 600 mm, about 250 mM to about 300 mm, about 275.1 mM to about 295.6 mM, about 280.3 mM to about 290.4 mM, about 285.9 mM to about 289.7 mM, about 286 mM to about 290 mm, about 287 mM to about 289 mm, about 292 mM to about 296 mm, about 293 mM to about 295 mm, about 513 mM to about 516 mm, about 514 mM to about 515 mm, about 288 mM, about 293.5 mM, about 294 mM, about 500.1 mM to about 520 mM, about 511 mM, about 514.5 mM, about 516.2 mM, or about 520 mM.

In some aspect, the protein excipient for use in the pharmaceutical composition described herein comprises silk fibroin, casein, a gelatin, an albumin, a yeast protein or combinations thereof. The protein excipient is additional protein added to the formulation to stabilize the active ingredient (e.g., virus or viral vector) and does not include the concentration of protein contributable from the active ingredient, such as a virus or viral vector. As used herein “silk fibroin” includes silkworm fibroin or spider silk protein. Any type of silk fibroin may be used in the compositions described herein. As used herein “albumin” includes a sterile nonpyrogenic preparation of serum albumin, most commonly obtained from human or bovine sources. Albumin from an egg may also be present. Albumin may also be recombinant albumin and may be from an animal-free source, e.g., isolated from yeast or a genetically engineered plant, such as rice. In some aspects the albumin comprises a human serum albumin or a bovine albumin. In one aspect, the protein is a human serum albumin.

In some aspects, the protein excipient may be present in the pharmaceutical composition described herein in any amount between about 1 g/L to about 15 g/L, about 2 g/L to about 14 g/L, about 3 g/L to about 13 g/L, about 4 g/L to about 12 g/L, about 5 g/L to about 11 g/L, about 4 g/L to about 11 g/L, about 6 g/L to about 10 g/L, about 7 g/L to about 9 g/L, about 3 g/L to about 7 g/L, about 4 g/L to about 6 g/L, about 4.5 g/L to about 5.5 g/L, about 4.75 g/L to about 5.25 g/L, about 4.85 g/L to about 5.15 g/L, about 8 g/L to about 12 g/L, about 9 g/L to about 11 g/L, about 9.5 g/L to about 10.5 g/L, about 9.75 g/L to about 10.25 g/L, about 9.7 g/L to about 9.9 g/L, or about 9.75 g/L to about 9.85 g/L, about 1 g/L, about 2 g/L, about 3 g/L, about 4 g/L, about 4.9 g/L, about 5 g/L, about 6 g/L, about 7 g/L, about 8 g/L, about 9 g/L, about 9.8 g/L, about 9.8 g/L, about 10.0 g/L, about 11.0 g/L, about 12.0 g/L, about 13.0 g/L, about 14.0 g/L, or of about 15.0 g/L.

In some aspects, no protein excipient is used in the pharmaceutical composition described herein. In some aspects, a protein excipient used in the pharmaceutical composition herein, is albumin (e.g., human serum albumin) and/or gelatin. In some aspects, a composition does not comprise, or does not contain a substantial or effective amount of, one or more of PEG8k, PS-80, lactalbumin, PS-20, or sodium metabisulfite. In some aspects, a composition does not comprise, or does not contain a substantial or effective amount of peptone, e.g., soy peptone.

In one aspect, the buffer for use in the pharmaceutical composition described herein comprises acetic acid, ammonium carbonate, ammonium phosphate, boric acid, citric acid, lactic acid, phosphoric acid, potassium citrate, potassium metaphosphate, potassium phosphate monobasic, potassium phosphate dibasic, sodium acetate, sodium citrate, sodium lactate solution, dibasic sodium phosphate, monobasic sodium phosphate, HEPES, MES, SSC, CP, Tris or combinations thereof. In some aspects, the buffer comprises HEPES, potassium phosphate monobasic, or potassium phosphate dibasic.

In some aspects, the buffer has a buffering capacity between pH 3 and pH 8, between pH 4 and pH 7.5, between pH 5 and pH 7, between pH 6 and pH 8, between pH 7 and pH 9. In another aspect, the buffering capacity is between pH of 6.0 to pH 6.5, pH 6.2 to pH 7.5, pH 7.0 to pH 7.9, pH 7.5 to pH 8.3, pH 8.0 to pH 8.5, about pH 6.0, about pH, 6.3, about pH, 6.5, about pH 6.7, about pH, 6.9, about pH 7.0, about pH 8.1, about pH 8.4, about pH 8.7, about pH 9.0, or about pH 9.5.

In some aspects, pharmaceutical composition described herein has a pH of about 7 to about 9.5, about 7.2 to about 9.5, about 7.4 to about 9, about 7.2 to about 8.5, about 7.2 to about 8.2, about 7.5 to about 8.3, about 7.3 to about 8.3, greater than 7.25, greater than 7.3, greater than 7.4, greater than or equal to 7.5, about 7, about 7.25, about 7.5, about 7.8, about 8, about 8.25, about 8.5, about 8.75, about 9, about 9.25 or about 9.5.

In some aspects, the buffer for use in the pharmaceutical composition described herein may be in an amount between about 2-25 mM, about 3-24 mM, about 4-23 mM, 5-22 mM, about 6-21 mM, about 7-20 mM, about 8-19 mM, about 9-18 mM, about 10-19 mM, about 11-18 mM, about 12-17 mM, about 13-16 mM, about 2 mM, about 3 mM, about 3 mM, about 4 mM, about 5 mM, about 6 mM, about 7 mM, about 7.5 mM, about 8.0 mM, about 9.0 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, or about 25 mM.

In one aspect the buffer may be in an amount between 1.0 g/L to about 10 g/L, about 2.0 g/L to about 9 g/L, about 3.0 g/L to about 8 g/L, about 4.0 g/L to about 8 g/L, about 5.0 g/L to about 9 g/L, about 6 g/L to about 8 g/L, about 1.0 g/L to about 3.0 g/L, about 1.1 g/L to about 2.9 g/L, about 1.2 g/L to about 2.7 g/L, about 1.3 g/L to about 2.5 g/L, about 1.5 g/L to about 2.3 g/L, about 1.5 g/L to about 2.9 g/L, about 1.8 g/L to about 3.0 g/L, about 2.0 g/L to about 2.8 g/L, about 1.1 g/L, about 1.2 g/L, about 1.5 g/L, about 1.8 g/L, about 2.1 g/L, about 2.4 g/L, about 2.6 g/L, about 2.8 g/L, about 3.0 g/L. In one aspect the buffer may be in an amount of about 1.0 g/L, about 2.0 g/L, about 3.0 g/L, about 4.0 g/L, about 5.0 g/L, about 6.0 g/L, about 7.0 g/L, about 8.0 g/L, about 9.0 g/L, or about 10.0 g/L.

In some aspects, the composition comprises a hepes buffer, but does not comprise either potassium phosphate monobasic or potassium phosphate dibasic. In some aspects, the composition comprises a hepes buffer, but does not comprise either potassium phosphate monobasic or potassium phosphate dibasic at a concentration higher than 0.01, 0.02, 0.03, 0.04, 0.05, 0.06, 0.07, 0.08, 0.09 or 0.1 g/L.

In some aspects, the composition comprises a potassium phosphate monobasic, potassium phosphate dibasic or both, but does not comprise a hepes buffer. In some aspects, the composition comprises a potassium phosphate monobasic, potassium phosphate dibasic or both, but does not comprise a hepes buffer at a concentration higher than 0.01, 0.02, 0.03, 0.04, 0.05, 0.06, 0.07, 0.08, 0.09 or 0.1 g/L.

In another embodiment described herein is a liquid pharmaceutical composition comprising the rVSV vectors described herein. In one aspect, the pharmaceutical composition comprises that shown in Table 2.

TABLE 2
Exemplary Compositions

| | | | | | |
|---|---|---|---|---|---|
| Exemplary Ingredients | A | B | C | D | E |

| | |
|---|---|
| EBS-LASV(rVSVn4Δg-LASVgpc1) | TCID₅₀/mL = 1 × 10⁵ − 1 × 10⁷ |

Sugars

| | | | | | |
|---|---|---|---|---|---|
| Trehalose (%) | 6.10 | 12.30 | 6.10 | — | — |
| Sucrose (%) | 0.15* | 0.15* | 12.40 | 0.15* | 0.15* |
| Mannitol (%) | — | 12.30 | 12.30 | 12.30 | — |
| Total Sugar | 6.10 | 24.60 | 30.8 | 12.30 | — |

Amino Acids

| | | | | | | | | | |
|---|---|---|---|---|---|---|---|---|---|
| Glutamic Acid (g/L) | 0.02 | g/L* | 0.02 g/L* | 0.02 | g/L* | 0.02 | g/L* | 0.02 | g/L* |

| | | | | | |
|---|---|---|---|---|---|
| Histidine (mM) | 141 | 147 | 147 | 147 | 147 |
| Threonine (mM) | 147 | 147 | — | 73.5 | 147 |
| Methionine (mM) | — | — | — | 147 | — |
| Glycine (mM) | — | — | 147 | 147 | — |
| Total Amino Acid | 288 | 294 | 294 | 514.5 | 294 |

Protein

| | | | | | |
|---|---|---|---|---|---|
| Recombinant Human Serum | 9.8 | 4.9 | — | — | — |
| Albumin (g/L) | | | | | |
| Total Protein | 9.8 | 4.9 | — | — | — |

Buffer

| HEPES (mM) | — | | — | 24.5 | | — | | — | |
|---|---|---|---|---|---|---|---|---|---|
| Potassium phosphate | 1.2 | | 0.01* | 0.01* | | 1.2 | | 1.2 | |
| monobasic (g/L) | | | | | | | | | |
| Potassium phosphate dibasic (g/L) | 2.8 | | 0.03* | 0.03* | | 2.8 | | 2.8 | |
| Total Buffer | 4.0 | g/L | 0 | 24.5 | mM | 4.0 | g/L | 4.0 | g/L |

*A simple dilution of drug product (DP) formulation into drug substance (DS) sucrose phosphate glutamate (SPG) at 2:98 ratio was performed and a small remaining amount of SPG content from DS remained during DP formulation manufacturing process.

It is understood that while Table 2 describes the formulation using a LASV transgene, the same formulations may be formulated using any or none of the viral transgenes described herein, including the MARV transgene, the EBOV transgene, or the SUDV transgene.

In another embodiment, the pharmaceutical composition described herein, comprises an rVSV having a TCID₅₀/mL=1×10⁵-1×10⁷; trehalose in an amount of about 5% to about 15%; sucrose in an amount of about 5% to about 15%; mannitol in an amount of about 5% to about 15%; glutamic acid in an amount of about 0.1 g/L to about 0.05 g/L; histidine in an amount of about 1 mM to about 200 mM; threonine in an amount of about 1 mM to about 200 mM; methionine in an amount of about 1 mM to about 200 mM; glycine in an amount of about 1 mM to about 200 mM.

In some aspects, a composition does not comprise one or more, or does not contain a substantial or effective amount, of the following excipients: 2-OH propyl β-CD, a-cyclodextrin, alanine, arginine, aspartic acid, citrulline, cysteine, dextran, dextrose, edta, gelatin, glutathione, lactalbumin, leucine, lysine, myo-inositol, PEG8k, phenylalanine, plasdone C-17, pluronic F68, proline, PS-20, PS-80, serine, sodium bicarbonate, sodium citrate, sodium metabisulfite, sorbitol, soy peptone, TRIS-HCl, tryptophan, or valine. In some aspects, a composition does not comprise one or more, or does not contain a substantial or effective amount, of the following excipients: glutamic acid, glycine, HEPES, potassium phosphate, threonine, or trehalose.

In some aspects, a composition does not comprise a chelator. In some aspects, a composition does not comprise one or more, or does not contain a substantial or effective amount, of Na-citrate, EDTA, or meta-bisulfite.

Stability of the pharmaceutical formulations, in particular liquid formulations as vaccines for global infectious disease is critical. Stable compositions that may endure temperature fluctuations and lengthy storage conditions is desirable, and makes a significant impact on efforts to control disease. In some aspects, the pharmaceutical composition comprises a TCID₅₀ log titer of the rVSV vector of between about log 4 to about log 8 at 2-8° C. for 5 months. In yet another aspect, the pharmaceutical composition comprises a TCID₅₀ log titer of the rVSV vector of between about log 5 to about log 7 at 2-8° C. for 5 months. In yet another aspect, the pharmaceutical composition comprises a TCID₅₀ log titer of the rVSV vector of about log 6 at 2-8° C. for 5 months. In some aspects, the pharmaceutical composition comprises a TCID₅₀ log titer of the rVSV vector of between about log 4 to about log 8 at 2-8° C. for 12 months. In yet another aspect, the pharmaceutical composition comprises a TCID₅₀ log titer of the rVSV vector of between about log 5 to about log 7 at 2-8° C. for 12 months. In yet another aspect, the pharmaceutical composition comprises a TCID₅₀ log titer of the rVSV vector of about log 6 at 2-8° C. for 12 months.

In another aspect, the pharmaceutical composition is stable at 2 to 8° C., at −15° C. to −25° C., at −18° C. to −22° C., at −20° C. to −25° C., at −15° C. to −25° C., less than −15° C., less than −18° C., less than −20° C., and at about −20° C.

In some aspects, the pharmaceutical composition maintains a TCID₅₀ log titer of the rVSV vector of between about log TCID₅₀ 4 per ml to about log 8 per ml at −20° C. for 5 months, 6 months, 7 months, 8 months, 9 months, 10 months, 11 months, or 12 months. In yet another aspect, the pharmaceutical composition comprises a TCID₅₀ log titer of the rVSV vector of between about log 5 per ml to about log 7 per ml at −20° C. for 5 months, 6 months, 7 months, 8 months, 9 months, 10 months, 11 months, or 12 months. In yet another aspect, the pharmaceutical composition comprises a TCID₅₀ log titer of the rVSV vector of about log 6 per ml at −20° C. for 5 months, 6 months, 7 months, 8 months, 9 months, 10 months, 11 months, or 12 months. In yet other aspects, after storage of the pharmaceutical composition at 2-8° C. or at −20° C. for 0.5, 1, 2, 3, or 4, 5, 6, 7, 8, 9, 10, 11 or 12 months, the difference in TCID₅₀ log titer is not more than 0.1. 0.2, 0.3, 0.4, 0.5, 0.6, 0.7, 0.8, 0.9, 1.0 or 1.1 log per ml less than the TCID₅₀ log titer at the time of being placed into storage. In one aspect, the pharmaceutical composition described herein comprises a TCID₅₀ log titer loss of no more than about 0.1. 0.2, 0.3. 0.4, 0.5, 0.6, 0.7, 0.8, 0.9, or 1.0 log after storage at 2-8° C. or at −20° C. for 1 month, 2 months, 3 months, 4 months, 5 months, 6 months, 7 months, 8 months, 9 months, 10 months, 11 months, or 12 months.

In another aspect, the pharmaceutical composition maintains a TCID₅₀ log titer of the rVSV vector or virus of between about log TCID₅₀ 4 per ml to about log 8 per ml at −20° C. for about 1 day to about 30 days, about 3 to about 15 days. In another aspect, the pharmaceutical composition maintains a TCID₅₀ log titer of the rVSV vector of between about log TCID₅₀ 4 per ml to about log 8 per ml-20° C. for about 1 day, about 7 days, about 14 days or about 30 days. In another aspect, the pharmaceutical composition maintains a TCID₅₀ log titer of the rVSV vector of between about log TCID₅₀ 4 per ml to about log 8 per ml at 2-8° C. for about 1 day to about 30 days, about 3 to about 15 days. In another aspect, the pharmaceutical composition maintains a TCID₅₀ log titer of the rVSV vector of between about log TCID₅₀ 4 per ml to about log 8 per ml at 2-8° C. for about 1 day, about 7 days, about 14 days or about 30 days. In another aspect, the pharmaceutical composition maintains a TCID₅₀ log titer of the rVSV vector of between about log TCID₅₀ 4 per ml to about log 8 per ml at 25° C. for about 1 day to about 30 days, about 3 to about 15 days. In another aspect, the pharmaceutical composition maintains a TCID₅₀ log titer of the rVSV vector of between about log TCID₅₀ 4 per ml to about log 8 per ml at 25° C. for about 1 day, about 3 days, about 5 days, about 7 days, or about 9 days.

In another aspect, the pharmaceutical composition maintains a TCID₅₀ log titer of the rVSV vector of between about log TCID₅₀ 4 per ml to about log 8 per ml after thawing at room temperature for up to about 10 days to about 30 days, In another aspect, the pharmaceutical composition maintains a TCID₅₀ log titer of the rVSV vector of between about log TCID₅₀ 4 per ml to about log 8 per ml after thawing at room temperature for up to about 10 days. In another aspect, the pharmaceutical composition maintains a TCID₅₀ log titer of the rVSV vector of between about log TCID₅₀ 4 per ml to about log 8 per ml after thawing at room temperature for up to about 20 days, In another aspect, the pharmaceutical composition maintains a TCID₅₀ log titer of the rVSV vector of between about log TCID₅₀ 4 per ml to about log 8 per ml after thawing at room temperature for up to about 30 days,

In another aspect, the pharmaceutical composition maintains a TCID₅₀ log titer of the rVSV vector of between about log TCID₅₀ 4 per ml to about log 8 per ml after thawing at room temperature, or for a single freeze-thaw cycle. In another aspect, the pharmaceutical composition maintains a TCID₅₀ log liter of between about log TCID₅₀ 4 per ml to about log 8 per ml after the rVSV vector after thawing at room temperature after two freeze-thaw cycles. In another aspect, the pharmaceutical composition maintains a TCID₅₀ log titer of between about log TCID₅₀ 4 per ml to about log 8 per ml after the rVSV vector after thawing at room temperature after three freeze-thaw cycles. In another aspect, the pharmaceutical composition maintains a TCID₅₀ log titer of between about log TCID₅₀ 4 per ml to about log 8 per ml after the rVSV vector after thawing at room temperature after four or more freeze-thaw cycles.

Another aspect of the present disclosure is a method of treating, inhibiting or preventing an infection caused by a virus in a subject in need thereof, comprising administering to the subject an effective amount of any of the pharmaceutical compositions described herein. In one aspect, the virus may be any virus. In another aspect, the virus may be an arenavirus. In another aspect the virus may be a Lassa virus. In another aspect the virus may be a filovirus. In another aspect, the virus may be a Marburg virus. In another aspect, the virus may be an Ebola virus. In another aspect, the virus may be a Sudan virus.

Another aspect described herein is a method of eliciting an immune response in a subject in need thereof, comprising administering to the subject an effective amount of any of the pharmaceutical compositions described herein. In one aspect, the immune response comprises inducing antibody formation or T cell or B-cell responses. In another aspect, the antibody is an anti-LASV antibody. In another aspect, the antibody is an anti-MARV antibody. In another aspect, the antibody is an anti-EBOV antibody, In another aspect, the antibody is an anti-SUDV antibody, In one aspect, the subject is a human or non-human mammal.

The pharmaceutical composition described herein may be administered with a pharmaceutically acceptable carrier using any effective conventional dosage unit forms, including, for example, as an injectable formulation. The composition may be injected or given intravenously (by IV) directly into a specific tissue in the body, where it is taken up by individual cells. The composition of the present disclosure may be administered intravenously, intra-arterially, intra-tumorally, intra-articularly to a joint, subcutaneously, or via intraperitoneal administration, or as local, regional, systemic, or continual administration. Thus, in one aspect described herein, the administration is intramuscular injection. In some aspects, the administration is localized. In some aspects, the injection may be subcutaneous injection, intravenous injection or parenteral injection.

The pharmaceutical compositions for the administration of the rVSV vector of this disclosure may conveniently be presented in unit dosage form and may be prepared by any of the methods well known in the art of pharmacy. In one aspect, the pharmaceutical composition may be formulated as a single-dose form. In another aspect, the pharmaceutical composition may be formulated as multi-dose form.

All methods include the step of bringing the active ingredient into association with liquid carrier which constitutes one or more accessory ingredients. In some aspects, prior to administration, the composition may be diluted with a diluent comprising the same inactive excipients as the composition or with sterile water. In some aspects, the composition may be diluted, e.g., to a minimum of 10¹ TCID 50/ml, or as appropriate. In general, the pharmaceutical compositions are prepared by uniformly and intimately bringing the active ingredient into association with a liquid carrier, and then, if necessary, shaping the product into the desired formulation. In the pharmaceutical composition, the active virus or viral (e.g., rVSV) vector is included in an amount sufficient to produce the desired effect upon the process or condition of diseases.

The virus or viral (e.g., rVSV) vector described herein may be administered with a pharmaceutically-acceptable carrier using any effective conventional dosage unit forms, including, for example, as an injectable formulation. The vector may be injected or given intravenously (by IV) directly into a specific tissue in the body, where it is taken up by individual cells. Thus, the compositions of the present disclosure may be administered intravenously, intra-arterially, intra-tumorally, intra-articularly to a joint, subcutaneously, or via intraperitoneal administration, or as local, regional, systemic, or continual administration. Thus, in one aspect described herein, the administration is intramuscular injection. In one aspect, the administration is localized.

### EXAMPLES

### Example 1—Vector Design and Construction

EBS-LASV is a live attenuated, replication competent, rVSV-vectored vaccine also referred to as rVSVn4Δg-LASVgpc1. EBS-LASV was constructed using known molecular biology techniques. The vector was engineered to include insertion of a human codon-optimized full-length LASV gpc (glycoprotein precursor) gene (Josiah strain, lineage IV) at position 1 of the VSV genome. Expression of recombinant gpc gene results in LASV GP on the surface of the rVSV virion designed as the active ingredient to induce a host-derived immune response against LASV. Furthermore, gene shuffling techniques were used to translocate the “N” gene for the nucleocapsid protein to position 4 of the VSV genome and to delete the wild-type “G” gene for the VSV glycoprotein. FIG. 1 describes the EBS-LASV vector construct.

### Example 2—Tissue Culture Infectious Dose (TCID₅₀) Assay

TCID₅₀ method for EBS-LASV. In order to measure infectivity of the EBS-LASV vector described herein, a viral replication assay was performed to determine the appropriate titer. In a 96-well plate, Vero cells were seeded at a density of 4000 cells/well supplemented in 1×DMEM containing 10% FBS and 1 mM sodium pyruvate. Different dilutions of between 10¹ to 10¹⁰ EBS-LASV in 100 μL in 1×DMEM with 1 mM sodium pyruvate were added to the wells and incubated for seven days at 37° C. with 5% CO₂. After seven days, the infectivity was determined by immunostaining with mouse anti-LASV monoclonal primary and goat anti-mouse horseradish peroxidase conjugate secondary antibodies. TCID₅₀ titers were determined by the Spearman-Karber formula. In the analysis, untreated cells were used as a negative control. For each assay, eight different wells per dilution per treatment group were assessed.

### Example 3—Formulation Stability

A number of potential excipients were evaluated for their effects on liquid formulation stability as well as on other criteria. Formulation stability was evaluated at various temperatures over various durations by a TCID₅₀ assay that could be used in empirical approaches as described herein. Temperatures most commonly tested included −80° C., −20° C., 2-8° C., 25° C., and 40° C. Durations most commonly initially tested included 1 day, 3 day, 7 day, 14 day, and 30 day, but also extended to as long as 90 days and 4.5, 6, 9, 12, 18, 24, 36, 48 and 60 months days. Excipients tested included those listed in Table 3. Approximately 175-180 formulations were tested at multiple temperatures and multiple time points for stability of infectivity.

TABLE 3
Tested Excipients

| Excipient | Value or Range Tested | | Excipient Category |
|---|---|---|---|
| a-cyclodextrin | 2% | | Sugar, encapsulator |
| Alanine | 50 | mM | Amino Acid |
| Arginine | 100 | mM | Amino Acid |
| Aspartic acid | 5 | mM | Amino Acid |
| Citrulline | 100 | mM | Amino Acid |
| Cysteine | 80 | mM | Amino Acid |
| Dextran | 1% | | Sugar |
| Dextrose | 7.5% | | Sugar |
| EDTA | 2 | mM | Chelator |
| Gelatin | 0.2% | | Complex Protein |
| | | | Mixture (undefined) |
| Glutamic Acid | 7 | mM | Amino Acid |
| Glutathione | 0.3 | mM | Tripeptide |
| | | | (Antioxidant) |
| Glycine | 75 to 150 | mM | Amino Acid |
| HEPES | 10 to 25 | mM | Buffer |
| Histidine | 10 to 250 | mM | Amino Acid |
| Potassium Phosphate | 11 to 25 | mM | Buffer |
| HSA | 0.1 to 10 | g/L | Protein |
| Lactalbumin | 5% | | Protein |
| Leucine | 150 | mM | Amino Acid |
| Lysine | 100 | mM | Amino Acid |
| Mannitol | 3 to 12.5% | | Sugar |
| Methionine | 50 to 150 | mM | Amino Acid |
| Myo-inositol | 150 | mM | Sugar |
| PEG8k | 1% | | Polymer |
| Phenylalanine | 100 | mM | Amino Acid |
| Plasdone C-17 | 2 | mg/mL | Polymer |
| Pluronic F68 | 0.05% | | Poloxamer |
| Proline | 100 | mM | Amino Acid |
| PS-20 | 0.05% | | Surfactant |
| PS-80 | 0.05% | | Surfactant |
| Serine | 100 | mM | Amino Acid |
| Sodium Bicarbonate | 5 | mM | Buffer |
| Sodium citrate | 0.78 to 1 | mM | Buffer |
| Sodium metabisulfite | 50 | mM | Salt (Antioxidant) |
| Sorbitol | 100 | mM | Sugar |
| Soy Peptone | 5 to 15 | mg/mL | Complex Protein |
| | | | Mixture (undefined) |
| Sucrose | 6.25 to 12.5% | | Sugar |
| Threonine | 75 to 150 | mM | Amino Acid |
| Trehalose | 6.25 to 12.5% | | Sugar |
| TRIS-HCl | 10 | mM | Buffer |
| Tryptophan | 20 | mM | Amino Acid |
| Valine | 100 | mM | Amino Acid |

### Example 4—Preliminary Excipient Examination

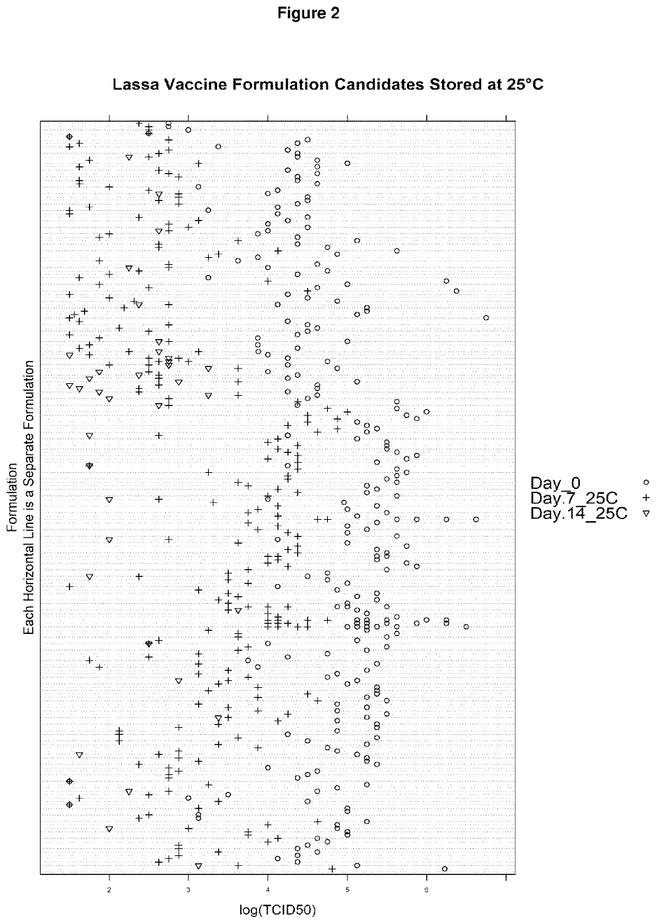

In order to gain an understanding of the stability of the active ingredient and potential effects of various classes of excipients, a number of studies were conducted with excipients added individually to the drug substance to examine the effects on critical quality attributes, such as stability at room temperature (25° C.). These excipients included those listed in Table 4 and some results are shown in FIG. 2.

TABLE 4
Excipients for Preliminary Experiments.

| Sugar | Buffer | Amino Acid | Chelator | pH | Other |
|---|---|---|---|---|---|
| Sucrose | Potassium | Histidine | Na-citrate | 6.0 | HSA |
| Trehalose | Phosphate | Methionine | EDTA | 6.5 | PS-20 |
| Sorbitol | Tris-HCl | Glutathione | Meta-bisulfite | 7.0 | PS-80 |
| Dextrose | Sodium | Glycine | | 7.5 | Pluronic F68 |
| Mannitol | bicarbonate | Arginine | | 8.0 | PEG-8000 |
| | HEPES | Lysine | | | Lactalbumin |
| | | Alanine | | | Plasdone |
| | | Proline | | | Myo-inositol |
| | | Cysteine | | | α-Cyclodextrin |
| | | Tryptophan | | | 2-OH propyl β-CD |
| | | Serine | | | |
| | | Leucine | | | |
| | | Valine | | | |
| | | Isoleucine | | | |
| | | Phenylalanine | | | |
| | | Threonine | | | |

### Statistical Analysis

Excipients consisting of buffer agents, lyo/cryoprotectants, solubilizing agents, and non-animal origin stabilizers shown in Table 4, were used during the initial stages of formulation development. Over 130 formulations consisting of different permutations of excipients were placed on stability and tested by TCID₅₀ assay. A Supervised Learning method, including Multiple Linear Regression with Forward, Backward, and Mixed Model Selection, was employed to gain further qualitative understanding of the stabilizing/destabilizing effect of each of 40+ excipients individually as well as that of each excipient pair resulting in a small set of excipients chosen for a Design of Experiments (DOE), discussed below. The excipients chosen produced stable results even when in combinations not used in the DOE.

Using stability data at 25° C. for seven days as criteria, Table 5 highlights the excipients that produced positive and negative effects with EBS-LASV. Some of these excipients were used in a DOE study.

TABLE 5
Contributions to stability (positive and negative) from statistical
analysis conducted before the DOE on all available data conducted
with excipients in an intermediate base buffer(s).

| Excipient Type | Positive | Negative |
|---|---|---|
| Sugars | Mannitol, Sucrose, | a-cyclodextrin, Dextran, |
| | Trehalose | Sorbitol |
| Amino Acids | Histidine, Citrulline, | Phenylalanine, Lysine, |
| | Serine, Glycine, Valine | Arginine, Cysteine, |
| | | Aspartic acid, Glutamic |
| | | acid, Leucine, Tryptophan |
| Other | Gelatin, HSA | PEG8k, PS-80, |
| | | Lactalbumin, PS-20, |
| | | Sodium metabisulfite |

### Design of Experiments (DOE)

A DOE study was conducted using a fractional factorial design with two levels for each excipient included in the study. This DOE examined the effects of two sugars, two pH buffers, and multiple stabilizers (including amino acids) with respect to improvement in stability (25° C.) and real-time stability at two temperatures (−20° C., 2-8° C.). Over 100 samples were tested in this DOE and analyzed using JMP® statistical software to obtain quantitative values for the contribution of each excipient and for two-factor interactions.

The DOE was designed to evaluate the contribution of each excipient to potency as measured by infectivity assay resulting in a TCID₅₀ value for the targeted storage temperatures of 5° C. (2-8° C.) and −20° C. for up to 90 days. A prediction profiler using JMP® can maximize the optimal desirability of each excipient, predict shelf-life and build a general model of stability analysis. Desirability function of the profiler shows the optimal concentration of each excipients to match the highest potency of the given temperature, with 1 being the most desirable.

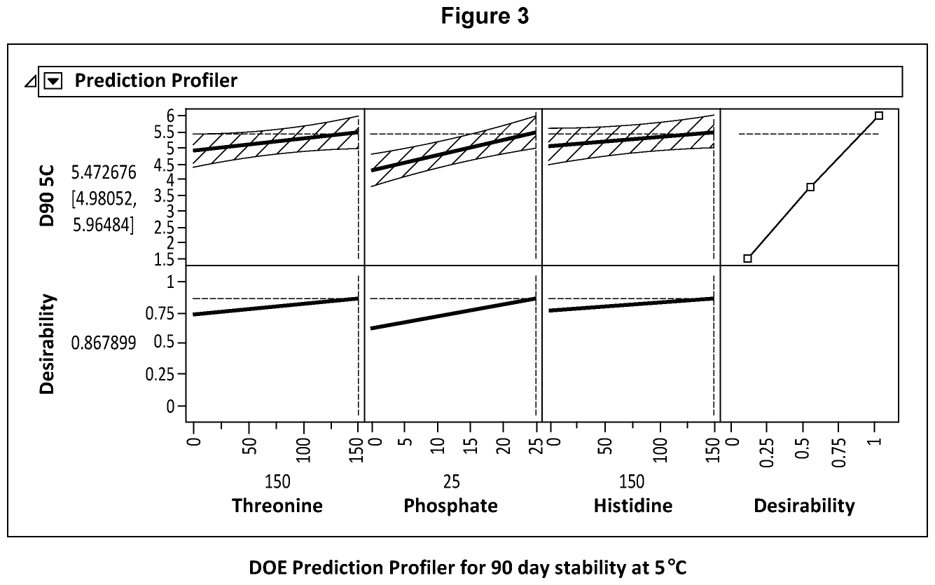

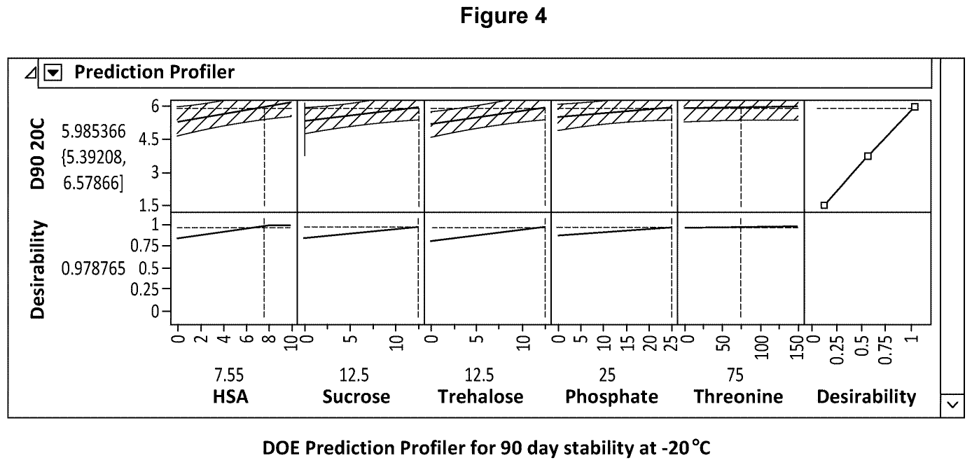

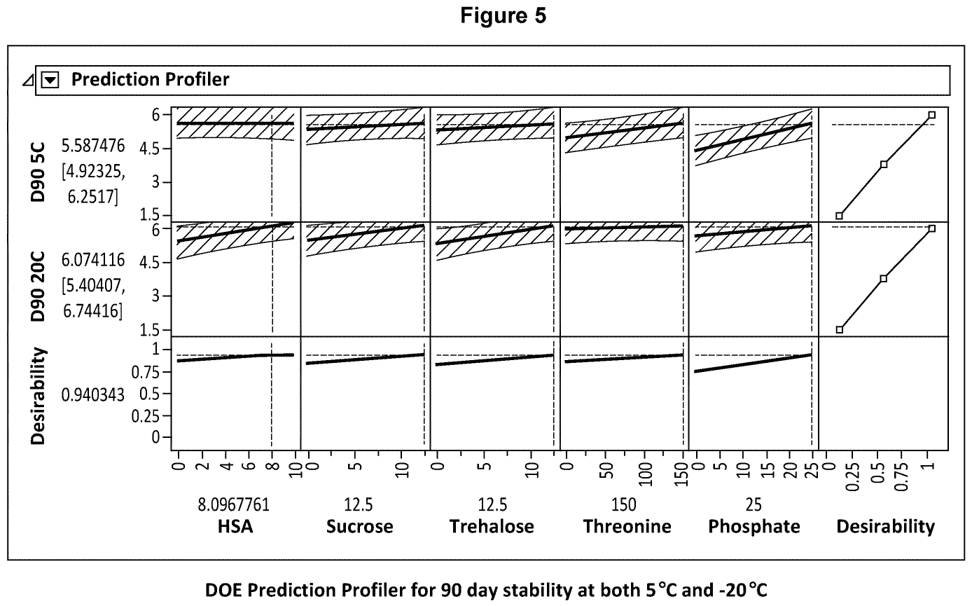

The results are depicted in FIGS. 3-5, where the x-axis is the concentration of each excipient, while the y-axis is the potency at 90 days at either 5° C. and/or −20° C. The results suggested that 5° C. and −20° C. had different degradation pathways as different excipients appeared to be responsible for stability at different temperature. Thus, the amino acids and buffer are the primary factors at stabilizing the rVSV-Lassa antigen at 5° C. (FIG. 3), while HSA and cryoprotectant sugars stabilize the frozen antigen at −20° C. (Error! Reference source not found.). The combination of all of these excipients stabilizes the rVSV when the DOE examined the effects of the excipients on stability as measured at each of the two temperatures (Error! Reference source not found.).

### Example 5—Assessment of HSA and Other Alternative Stabilizer Formulations

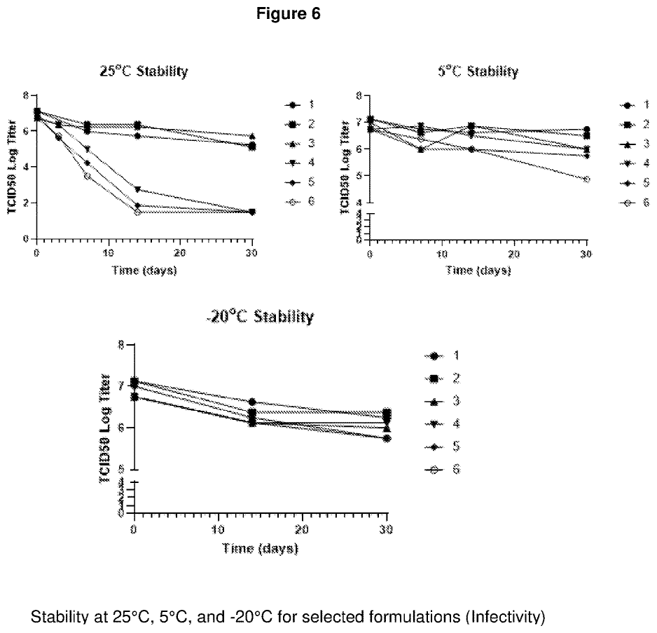

Formulations with different concentrations of HSA or soy peptone alone were assessed for short-term stability. The EBS-LASV vector was prepared in formulations shown in Error! Reference source not found. with 2×10⁷ TCID₅₀/mL of EBS-LASV. The formulations were filled into 2R glass vials at volume of 0.75 mL and placed on stability at −20° C., 5° C., and 25° C. At days 0, 7, 14, and 30, samples were pulled and potency was analyzed by TCID₅₀ assay. As shown in FIG. 6, the results show that formulations (1 (also referred to as Formulation A), 2) with HSA had reduced titer loss when stored at both −20° C. and 5° C. for at least 30 days. Formulations (4, 5, 6) with soy peptone had increased titer loss at −20° C. and 5° C. after 30 days with complete loss of infectivity at 25° C. after 30 days at all soy peptone concentration range. No change in pH, osmolality, and appearance were observed for all samples.

TABLE 6
Example 4 - Formulation Design

| | Potassium | | | | | Glutamic | | Soy |
|---|---|---|---|---|---|---|---|---|
| | Phosphate | Trehalose | Sucrose | Histidine | Threonine | acid | HSA | Peptone |
| Formulation | (mM) | (%) | (%) | (mM) | (mM) | (mM) | (g/L) | (g/L) |
| A | 24.7 | 6.1 | 0.15 | 141 | 147 | 0.1 | 9.8 | 0 |
| 2 | 24.7 | 6.1 | 0.15 | 141 | 147 | 0.1 | 1.0 | 0 |
| 3 | 24.7 | 6.1 | 0.15 | 141 | 147 | 0.1 | 0 | 0 |
| 4 | 24.7 | 6.1 | 0.15 | 141 | 147 | 0.1 | 0 | 4.9 |
| 5 | 24.7 | 6.1 | 0.15 | 141 | 147 | 0.1 | 0 | 9.8 |
| 6 | 24.7 | 6.1 | 0.15 | 141 | 147 | 0.1 | 0 | 14.7 |

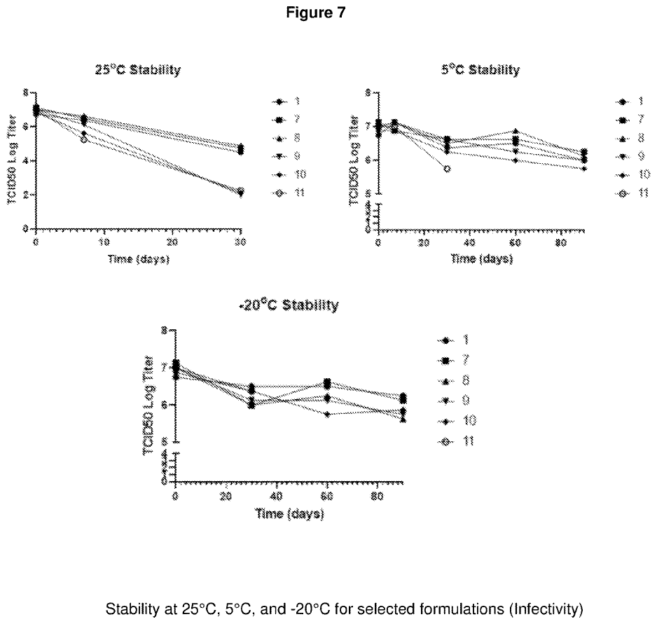

Another study was performed to evaluate formulations of different concentration of HSA and soy peptone in combination. As shown in Table 7, the EBS-LASV vector was prepared at 2×10⁷ TCID₅₀/mL EBS-LASV and filled at 0.75 mL. The samples were placed on stability −20° C., 5° C., and 25° C. At days 0, 7, 30, 60, and 90, samples were pulled, and the potency was determined by TCID₅₀. Formulations A and 7 with 9.8 g/L and 4.9 g/L HSA, respectively, outperformed all formulations containing soy peptone and/or lower concentration of HSA at −20° C. after 90 days (FIG. 7). Formulations containing any soy peptone performed much worse than the formulations without soy peptone at 25° C.

TABLE 7
Example 4 - Formulation Design

| | Potassium | | | | | Glutamic | | Soy |
|---|---|---|---|---|---|---|---|---|
| | Phosphate | Trehalose | Sucrose | Histidine | Threonine | acid | HSA | Peptone |
| Formulation | (mM) | (%) | (%) | (mM) | (mM) | (mM) | (g/L) | (g/L) |
| A | 24.7 | 6.1 | 0.15 | 141 | 147 | 0.1 | 9.8 | 0 |
| 7 | 24.7 | 6.1 | 0.15 | 141 | 147 | 0.1 | 4.9 | 0 |
| 8 | 24.7 | 6.1 | 0.15 | 141 | 147 | 0.1 | 0.1 | 0 |
| 9 | 24.7 | 6.1 | 0.15 | 141 | 147 | 0.1 | 4.9 | 4.9 |
| 10 | 24.7 | 6.1 | 0.15 | 141 | 147 | 0.1 | 1.0 | 8.8 |
| 11 | 24.7 | 6.1 | 0.15 | 141 | 147 | 0.1 | 0.1 | 9.7 |

### Example 6—pH Effect on the EBS-LASV

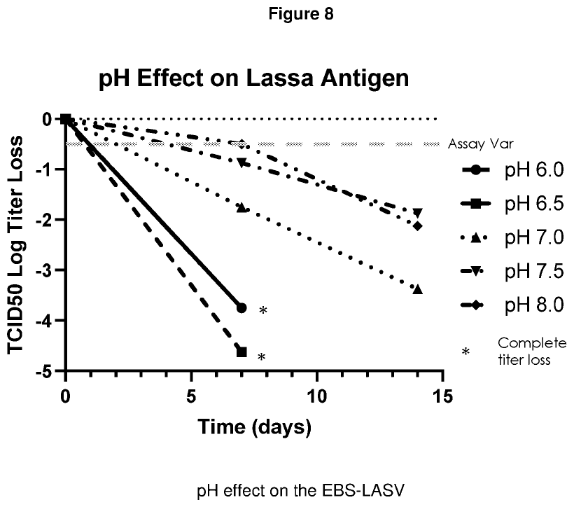

The pH effect on potency (titer) of the EBS-LASV the Lassa antigen was evaluated between pH 6.0 to pH 8.0 (FIG. 8). Formulations containing sucrose, phosphate buffer, and glutamic acid were pH-adjusted with sodium hydroxide to pH 6.0, 6.5, 7.0, 7.5, and 8.0. The formulations were placed on stability at 25° C. for up to 14 days. TCID₅₀ results indicated that the virus at pH 6.0 and 6.5 had complete loss of infectivity within 7 days, while virus at pH 7.5 and 8.0 maintained less than 1 log loss after 7 days and less than 2 log loss after 14 days, similar to other suitable formulations at pH 7.4. Therefore, at least for the conditions tested here, EBS-LASV is dependent on pH and should be a pH higher than pH 6.5 to maintain stability.

### Example 7—Freeze-Thaw Testing

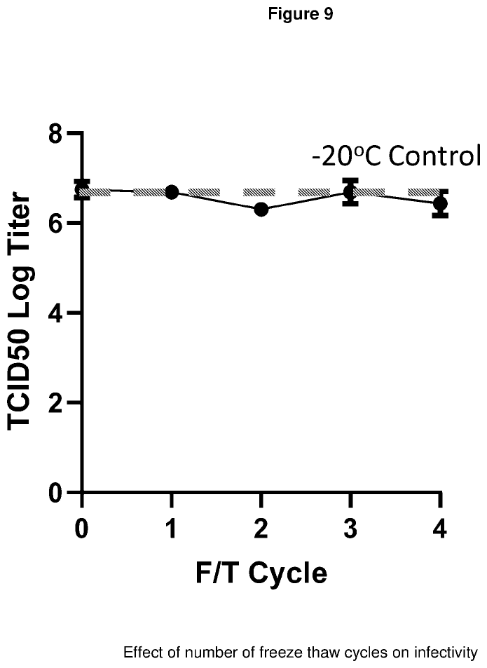

A freeze-thaw study was performed to evaluate the effectiveness of cryoprotectant to prevent or reduce the loss of potency after multiple freeze-thaws. Briefly, EBS-LASV in Formulation 1 described in Table 7 was subjected to 4 cycles of freeze-thaw from −20° C. to RT. The potency was analyzed by TCID₅₀ assay. As shown in FIG. 9, the potency did not show meaningful difference for at least 4 freeze-thaw cycles.

### Example 8—Stability to Slow Thaw (on Ice at Room Temperature)

Most, if not all, VSV-based vaccines in commercialization and various stages of clinical development must currently be stored long-term as frozen. The thawing rate of live virus-based vaccines can play a large role on the potency, thus making the thawing process limited. In particular, ERVEBO® (Ebola Zaire Vaccine, Live) requires fast thawing at room temperature to maintain viral titer, while thawing at refrigerating condition is not feasible [presented by Lynne Isopi, Principal Scientist, Merck at American Drug Delivery and Formulation Summit 2021 on 13 Sep. 2021].

A thawing study from −20° C. to refrigerated (5° C.) or room temperature was evaluated with EBS-LASV. The vials were placed within cardboard boxes in a 7 by 7 configuration and stored at −20° C. After overnight storage, the boxes were transferred to 5° C. refrigeration (slow thawing) or at room temperature (fast thawing) until all the vials were completely thawed. Both boxes were then placed or remained at 5° C. for overnight, next day placed at −80° C. and later thawed at room temperature and the potency was analyzed by TCID₅₀. The negative control was an EBS-LASV sample continuously stored at −80° C. without thawing. As shown in Table 8, there were no potency differences between slow or fast thawing, despite slow thawing taking about three times longer to thaw. The vials placed in the inner boxes also thawed slower for both thawing temperatures but did not have an effect on the potency.

TABLE 8
EBS-LASV fast v. slow thawing

| Sample | Vial Format | Thawing Time | TCID₅₀ |
|---|---|---|---|
| Negative control: | 1 vial | N/A | 7.1 |
| stored at −80° C. | | | |
| Fast thaw: −20° C. → | 7 × 7 | 1 h | 6.9 |
| RT → 2-8° C. → −80° C. | | | |
| Slow thaw: −20° C. → | 7 × 7 | 3 h | 6.7 |
| 2-8° C. → −80° C. | | | |

### Example 9—Temperature Stability of the Formulations

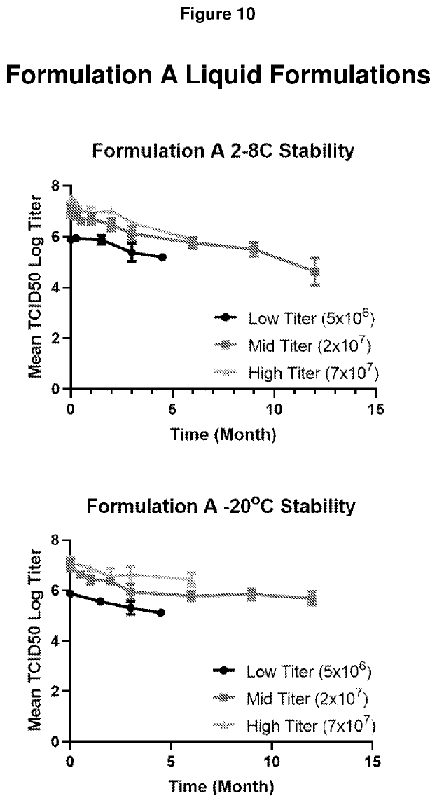

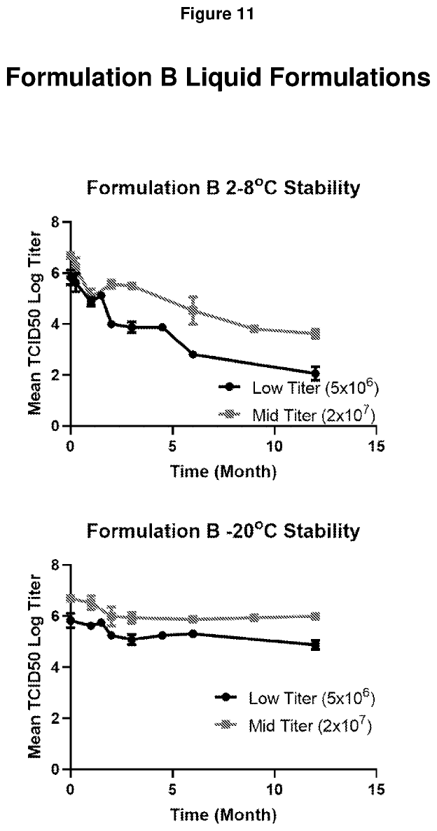

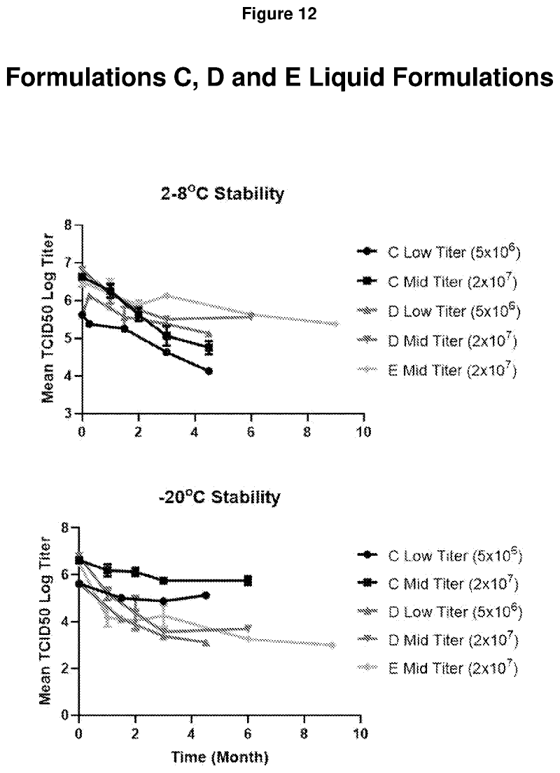

The formulations A, B, C, D, E, and F were subjected to stability testing with EBS-LASV as described above, at both 2-8° C. and −20° C. The results are described in FIGS. 10-12. Across low (5×10⁶ TCID₅₀/mL), middle (2×10⁷ TCID₅₀/mL), and high (7×10⁷ TCID₅₀/mL) titer range, Formulation A maintained stability at 2-8° C. for up to three months and −20° C. for at least 12 months and all replicates were consistent with assay variability. Across low (5×10⁶ TCID₅₀/mL) and middle (2×10⁷ TCID₅₀/mL) titer range, Formulation B exhibited stability at −20° C. for at least 12 months but not at 2-8° C., and all replicates were consistent with assay variability. Formulation C was stable at −20° C. for at least 6 months. Formulations D and E were stable at 2-8° C. for up to 3 months and at least 9 months, respectively. As predicted by the DOE experiments of Example 2, different excipients contributed to stability at different temperatures. Formulations B-E comprise excipients that conferred stability to the composition at their respective temperatures. However, Formulation A comprises the combination of excipients that confer stability at both 2-8° C. and −20° C. Thus, a combination of amino acids and buffers were found to confer stability at higher temperatures, while a combination of sugars and protein conferred stability at lower temperatures. Formulation A provides both of sets of excipients which were found to confer stability at both ends of the temperature spectrum over an extended period of time.

### Example 10—Formulations on Other rVSV-Based Vectors Expressing Other Heterologous Protein

Some of the excipients within the EBS-LASV formulation have been evaluated on other rVSV-based antigens. The excipients used here were not extensively optimized for a recombinant, replication competent, live-attenuated VSV containing the Marburg glycoprotein (EBS-MARV) and three separate rVSV pseudotypes containing Ebola (rVSV-EBOV), Sudan (rVSV-SUDV), and Marburg (rVSV-MARV) glycoproteins, respectively. Similar to EBS-LASV, the EBS-MARV (directed to encoding/expressing the Marburg virus glycoprotein) is engineered with attenuating modifications, including the translocation of the N gene from the first position to the fourth position (N4), truncation of the G protein cytoplasmic tail, and insertion of the Marburg glycoprotein. In contrast to EBS-LASV, EBS-MARV expresses the glycoprotein from Marburg virus, and additionally maintains and expresses the VSV glycoprotein gene, whereas the EBS-LASV has the VSV glycoprotein gene deleted. So the envelopes of the two viruses will differ in that the envelope of EBS-LASV has/displays the Lassa glycoprotein, whereas the EBS-MARV has/displays both the VSV and Marburg glycoproteins and not the Lassa glycoprotein. rVSV pseudotypes (rVSV-EBOV, rVSV-SUDV, and rVSV-MARV) were constructed by deleting and replacing the G protein of the VSV with the envelope glycoprotein of either Ebola, Sudan, or Marburg virus. Additionally, rVSV-EBOV, rVSV-SUDV, and rVSV-MARV contained a firefly luciferase gene. Expression of luciferase was used to measure infectivity. Unlike EBS-LASV and EBS-MARV, the rVSV-EBOV, rVSV-SUDV, and rVSV-MARV viruses do not have additional attenuating modifications, such as translocation of the N gene.

Two studies were performed to further characterize EBS-MARV:
1. Freeze-thaw
2. Stability at various temperatures

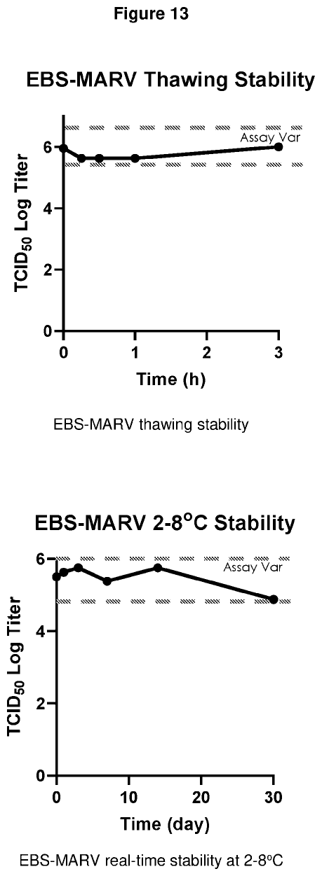

EBS-MARV in a formulation containing 10% sucrose, 0.1% Pluronic® F68, 58 mM histidine in 10 mM potassium phosphate buffer, with a starting viral titer of 1×10⁴ to 1×10⁶ TCID₅₀/mL, was taken from −80° C. and allowed to thaw at room temperature for 40 minutes. A small aliquot was taken once thawed and additionally 15, 30, 60, and 180 minutes post-thawed at room temperature. The samples were stored at −80° C. and analyzed by TCID₅₀. EBS-MARV remained stable post-thaw for 3 hours, with no drop in titer (FIG. 13A). The dotted lines in FIG. 13A represent a 0.5 log titer difference from T₀ time point, which is the assay variability of the TCID₅₀ assay.

The EBS-MARV formulation described above, and the rVSV-EBOV, rVSV-SUDV, and rVSV-MARV formulated to contain 10% sucrose, 0.1% Pluronic® F68, 58 mM histidine in 10 mM potassium phosphate buffer, with a starting viral titer of 1×10⁶ to 1×10⁸ TCID₅₀/mL, were additionally buffer exchanged into three formulation buffers and placed in freeze-thaw and stability studies (Table 9):

TABLE 9
Additional Buffer Formulations for EBS-MARV

| Formulation | Excipients |
|---|---|
| A | 6.1% trehalose, 141 mM histidine, 147 mM |
| | threonine, 9.8 g/L HSA, 25 mM |
| | potassium phosphate, 0.15% sucrose, |
| | 0.02 g/L glutamic acid, pH 7.4 |
| 2 | 2.5 g/L HSA, 10 mM TRIS, pH 7.2 |
| 3 | 7.5% sucrose, 7 mM glutamic acid, 11 mM |
| | potassium phosphate 0.2% gelatin, pH 7.4 |

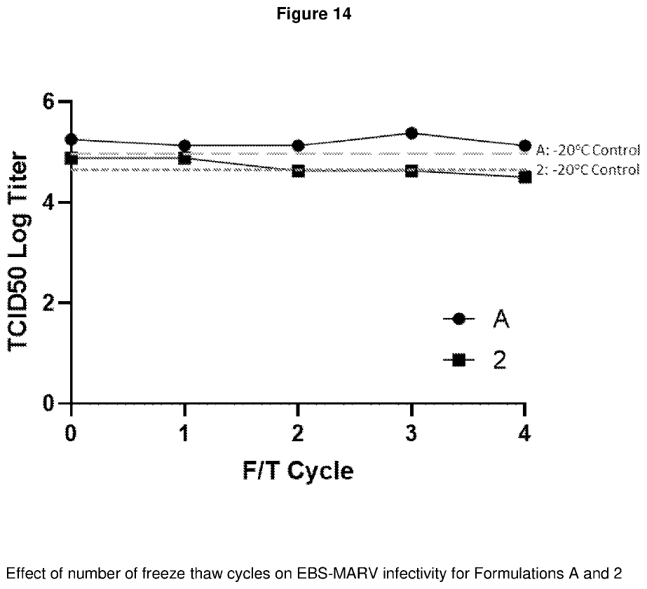

Formulation A was tested in previous examples. Formulation 2 is similar to the reported formulation for ERVEBO® vaccine, which is a live recombinant viral vaccine consisting of a vesicular stomatitis virus (VSV) backbone deleted for the VSV envelope glycoprotein and substituted with the envelope glycoprotein of the Zaire ebolavirus (ERVEBO® Package Insert, revised 07/2023). EBS-MARV, rVSV-EBOV, rVSV-SUDV, and rVSV-MARV, each in these three formulations, were subjected to 4 cycles of freeze-thaws from −20° C. to RT. The freeze-thaw samples were compared to control samples stored at −80° C. and −20° C. without freeze-thaw cycles. The potency was analyzed by TCID₅₀ assay for EBS-MARV or viral titration via luciferase activity for rVSV-EBOV, rVSV-SUDV, and rVSV-MARV. As shown in FIG. 14, the potency remained consistent on EBS-MARV infectivity for up to four freeze-thaw cycles on both Formulations A and 2. Table 10 summarizes the results of luciferase activity for rVSV-EBOV, rVSV-SUDV, and rVSV-MARV after four freeze-thaw cycles in Formulations A, 2, and 3. Each data point represents the mean relative luciferase unit (RLU) from three replicates. The pseudovirus titers for rVSV-EBOV, rVSV-SUDV, and rVSV-MARV remained stable after four freeze-thaw cycles for formulations A, 2, and 3.

TABLE 10
Effect of 4 Freeze-thaw Cycles on each rVSV
pseudotype in Formulations A, 2, and 3

| | | Relative Luciferase Unit (RLU) Log Titer | | |
|---|---|---|---|---|
| | | rVSV- | rVSV- | rVSV- |
| Formulation | Sample | EBOV | MARV | SUDV |
| A | After 4 freeze- | 7.124 | 7.795 | 7.176 |
| | thaw cycles | (−0.232) | (−0.028) | (0.036) |
| | −20° C. | 7.182 | 7.814 | 6.975 |
| | Control | | | |
| | −80° C. | 7.356 | 7.823 | 7.140 |
| | Control | | | |
| 2 | After 4 freeze- | 7.114 | 7.620 | 7.134 |
| | thaw cycles | (−0.185) | (−0.115) | (−0.022) |
| | −20° C. | 7.076 | 7.655 | 7.173 |
| | Control | | | |
| | −80° C. | 7.299 | 7.736 | 7.155 |
| | Control | | | |
| 3 | After 4 freeze- | 6.292 | 7.152 | 6.389 |
| | thaw cycles | (−0.415) | (−0.186) | (−0.295) |
| | −20° C. | 6.380 | 7.134 | 6.600 |
| | Control | | | |
| | −80° C. | 6.708 | 7.338 | 6.684 |
| | Control | | | |

NOTE:
Parentheses indicate the change in log RLU after four freeze-thaw cycles compared to the −80° C. control.

EBS-MARV, rVSV-EBOV, rVSV-SUDV, and rVSV-MARV in these three formulations were placed in conditions at −20° C. and 5° C. and stressed stability condition at 25° C. Table 11 summarizes the study design:

TABLE 11
EBS-MARV, rVSV-EBOV, rVSV-SUDV, and rVSV-
MARV DP storage conditions and time points

| | |
|---|---|
| Storage Temperatures | Time Points (days) ¹ |

| | | |
|---|---|---|
| −20° | C. | 7, 14 |
| 5 ± 3° | C. | 7, 14 |

| | |
|---|---|
| 25° C./60% RH | 1, 3, 7, 14 |

NOTE:
¹ Data from control sample at −80° C. from the freeze-thaw study is used as T = 0 data for stability studies. At the time of filing this application, Day 30 time point is ongoing.

NOTE: ¹Data from control sample at −80° C. from the freeze-thaw study was used as T=0 data for stability studies. At the time of filing this application, Day 30 time point is ongoing.

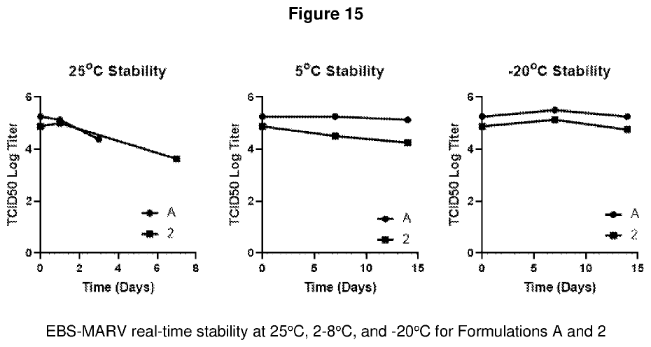

FIG. 15 summarizes the stability data of EBS-MARV with Formulations A and 2. Both Formulations A and 2 show stability of up to 1 day at 25° C. and at least 14 days at −20° C. For the 5° C. storage condition, Formulation A remained stable by TCID₅₀ for at least 14 days, while Formulation 2 had a stability loss of 0.625 log after 14 days with EBS-MARV. During the stability study, there was a bacterial contamination observed in samples at 25° C. for Formulation A at 7 day and 14 day and Formulation 2 at 3 day and 14 day. These samples are being re-analyzed. Additionally, Day 30 samples stored at 5° C. and −20° C. are pending analysis.

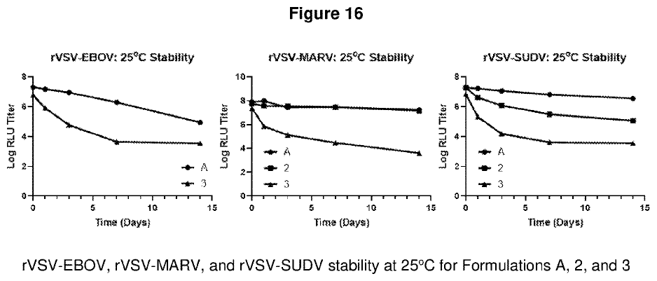

Table 12 summarizes the −20° C. and 5° C. and FIG. 16 describes the 25° C. stability data of rVSV-EBOV, rVSV-MARV, and rVSV-SUDV on Formulations A, 2, and 3. No loss in potency was observed for the three formulations stored at −20° C. for 14 days with the three rVSV pseudovirus. Formulation 2 RLU titers for samples stored at 5° C. for 14 days in all pseudotypes remained consistent. For 5° C. 14-day stability condition, Formulation 3 showed the largest titer drop-off compared to the other formulations with 1.5 to 2.6 log loss for the three rVSVs. Formulation A at 5° C. for 14 days had some stability loss of around 0.6 log loss for rVSV-EBOV and rVSV-MARV, while within the assay variability of 0.5 log for rVSV-SUDV. The 25° C. data and 30 day −20° C. and 5° C. data are ongoing.

For 25° C. stability on rVSV-EBOV, Formulation 3 was not stable at 1 day with 0.9 log drop in titer and 3.2 log drop off by 14 day, while Formulation A was stable for up to 3 day (<0.5 log drop), and had 2.4 log drop in titer by 14 days. Formulation 2 failed to meet the assay criteria and retesting is in progress. Formulation A and 2 stability were comparable for rVSV-MARV at 25° C. as both formulations were stable for up to 7 days and had 0.6 log drop after 14 days. In contrast, Formulation 3 was not stable for even a day at 25° C. and had 3.8 log drop in titer after 14 days. For 25° C. stability on rVSV-SUDV, Formulation A performed better than both Formulations 2 and 3 as Formulation A being stable for at least 7 days and had 0.7 log drop after 14 days. In contrast, both Formulations 2 and 3 were not stable for even a day at 25° C. on rVSV-SUDV with titer drop of 2.2 log and 3.3 log after 14 days, respectively.

TABLE 12
Stability on each rVSV pseudotype in Formulations A,
2, and 3 at −20° C. and 5° C.

| | Temperature | Time | Relative Luciferase Unit (RLU) Log Titer | | |
|---|---|---|---|---|---|
| Formulation | (° C.) | (Days) | rVSV-EBOV | rVSV-MARV | rVSV-SUDV |
| A | N/A | 0 | 7.324 | 7.928 | 7.260 |
| | −20 | 14 | 7.057 | 7.781 | 7.100 |
| | | | (−0.267) | (−0.147) | (−0.160) |
| | 5 | 14 | 6.731 | 7.274 | 6.884 |
| | | | (−0.594) | (−0.654) | (−0.376) |
| 2 | N/A | 0 | 7.316 | 7.856 | 7.185 |
| | −20 | 14 | 7.243 | 7.744 | 7.033 |
| | | | (−0.073) | (−0.112) | (−0.151) |
| | 5 | 14 | 7.660 | 7.635 | 7.167 |
| | | | (0.344) | (−0.221) | (−0.017) |
| 3 | N/A | 0 | 6.736 | 7.418 | 6.956 |
| | −20 | 14 | 6.551 | 7.279 | 6.663 |
| | | | (−0.185) | (−0.140) | (−0.293) |
| | 5 | 14 | 5.248 | 5.064 | 4.322 |
| | | | (−1.488) | (−2.354) | (−2.634) |

NOTE:
Parentheses indicate the change in log RLU when compared to T = 0.

EBS-MARV in 10% sucrose, 0.1% Pluronic® F68, 58 mM histidine in 10 mM potassium phosphate buffer was also placed in real-time stability at 2-8° C. for up to 30 days and the potency was analyzed by TCID₅₀. EBS-MARV remains stable through 14 days at 2-8° C. (titer drop <0.5 log) but had a titer loss of 0.625 log TCID₅₀ at 30 days (FIG. 9B). The dashed lines FIG. 13B represent a 0.5 log titer difference from TO time point, which is the assay variability of the TCID₅₀ assay.

EBS-MARV was buffer exchanged into two more formulation buffers (Table 11) and placed at 25° C. for up to 7 days:

TABLE 11
Additional Buffer Formulations for EBS-MARV

| Formulation | Excipients |
|---|---|
| 4 | 10% sucrose, 0.1% Pluronic, 58 mM histidine |
| | in 10 mM potassium phosphate, 0.2% gelatin, pH 7.4 |
| 5 | 6.3% sucrose, 1.47 mM mono-potassium |
| | phosphate, 7.2 g/L NaCl, 8.1 mM |
| | disodium phosphate, 0.2% gelatin, pH 7.4 |

All three formulations showed similar stability with 0.5 to 1 log loss after 7 days at 25° C. (Error! Reference source not found. 17), which is comparable with EBS-LASV formulations that demonstrated stability in previous Examples.

### Prophetic Examples

EBS-MARV, rVSV-EBOV, rVSV-SUDV, and rVSV-MARV in the three formulations described above are placed in conditions at −20° C. and 5° C. and stability will be observed at 30 days as well as for EBS-MARV in stressed stability condition at 25° C. for 14 days:

TABLE 11
EBS-MARV, rVSV-EBOV, rVSV-SUDV, and rVSV-
MARV DP storage conditions and time points

| | |
|---|---|
| Storage Temperatures | Time Points (days) ¹ |

| | | |
|---|---|---|
| −20° | C. | 30 |
| 5 ± 3° | C. | 30 |

| | |
|---|---|
| 25° C./60% RH | 14 (EBS-MARV only) |

NOTE:
¹ Data from control sample at −80° C. from the freeze-thaw study is used as T = 0 data for stability studies.

Stability data of rVSV-EBOV, rVSV-MARV, and rVSV-SUDV on Formulations A, 2, and 3 will be further studied at 25° C. data 14 day, and −20° C. and 5° C. at 30 days.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

1.-20. (canceled)

22.-50. (canceled)

51. A pharmaceutical composition comprising:
(a) about 5% to about 40% of one or more sugars;
(b) about 50 mM to about 600 mM of one or more amino acids;
(c) about 0.1 g/L to about 15 g/L of one or more protein; and
(d) a virus or viral vector.

52. The composition of claim 51, wherein the virus or viral vector is a Rhabdovirus vector.

53. The composition of claim 51, wherein the virus or viral vector is a Vesicular Stomatitis Virus (VSV) vector.

54. The composition of claim 51, wherein the virus or vector does not encode a homologous viral glycoprotein or fragment thereof.

55. The composition of claim 51, wherein the vector encodes a heterologous viral transgene.

56. The composition of claim 55, wherein the heterologous viral transgene is a Lassa virus transgene, a Ebola virus transgene, a Sudan virus transgene or a Marburg virus transgene.

57. The composition of claim 55, wherein the heterologous viral transgene is located at position of 1 of the viral genome.

58. The composition of claim 56, wherein a nucleocapsid N gene is located at position 4 of the viral genome.

59. The composition of claim 56, wherein the heterologous Lassa virus transgene encodes a Lassa virus glycoprotein or fragment thereof.

60. The composition of claim 56, wherein the heterologous Lassa virus transgene encodes a Lassa virus immunogen.

61. The composition of claim 55, wherein the heterologous viral transgene is a Marburg virus transgene.

62. The composition of claim 61, wherein the heterologous Marburg virus transgene encodes a Marburg virus glycoprotein or fragment thereof.

63. The composition of claim 61, wherein the heterologous Marburg virus transgene encodes a Marburg virus immunogen.

64. The composition of claim 55, wherein the heterologous viral transgene is an Ebola virus transgene.

65. The composition of claim 64, wherein the heterologous Ebola virus transgene encodes an Ebola virus glycoprotein or fragment thereof.

66. The composition of claim 64, wherein the heterologous Ebola virus transgene encodes a Sudan virus immunogen.

67. The composition of claim 55, wherein the heterologous viral transgene is a Sudan virus transgene.

68. The composition of claim 67, wherein the heterologous Sudan virus transgene encodes a Sudan virus glycoprotein or fragment thereof.

69. The composition of claim 67, wherein the heterologous Sudan virus transgene encodes a Sudan virus immunogen.

70. A method of treating, inhibiting or preventing an infection caused by a virus in a subject in need thereof, comprising administering to the subject an effective amount of the pharmaceutical composition of claim 51.

71. A method of eliciting an immune response in a subject in need thereof, comprising administering to the subject an effective amount of the pharmaceutical composition of claim 51.
